(19) United States
(12) Patent Application Publication
(10) Pub. No.: US 2019/0248830 A1
(43) Pub. Date: Aug. 15, 2019

(54) PROLIPOSOMAL TESTOSTERONE UNDECANOATE FORMULATIONS

(71) Applicants: WESTERN UNIVERSITY OF HEALTH SCIENCES, Pomona, CA (US); TESORX PHARMA, LLC, Menlo Park, CA (US)

(72) Inventors: Guru V. BETAGERI, Chino Hills, CA (US); Ramachandran THIRUCOTE, Atherton, CA (US); Veeran Gowda KADAJI, North Hollywood, CA (US); Natarajan VENKATESAN, Diamond Bar, CA (US)

(73) Assignees: Western University of Health Sciences, Pomona, CA (US); Tesorx Pharma, LLC, Menlo Park, CA (US)

(21) Appl. No.: 16/068,190

(22) PCT Filed: Jan. 9, 2017
(86) PCT No.: PCT/US2017/012739

§ 371 Date: Jul. 5, 2018

Related U.S. Application Data
(60) Provisional application No. 62/394,576, filed on Sep. 14, 2016.
(60) Provisional application No. 62/276,452, filed on Jan. 8, 2016.

(51) Int. Cl. C07J 9/00; A61K 9/00; A61K 9/127; A61K 9/48

(52) U.S. Cl. CPC C07J 9/005; A61K 9/0053; A61P 1/16; A61K 9/4866; A61K 9/1277

(57) ABSTRACT

Novel testosterone undecanoate (TU) formulations are disclosed in which TU is incorporated into proliposomal powder dispersions of TU and distearoyl phosphatidylcholine (DSPC). The proliposomal powder dispersions of the invention can also be combined with pharmaceutically acceptable excipients, and incorporated into enterically coated oral dosage forms that are useful for testosterone replacement therapy.

### CROSS-REFERENCE TO RELATED APPLICATIONS

This application claims priority to U.S. Application Ser. No. 62/276,452, filed 8 Jan. 2016, and to U.S. Application Ser. No. 62/394,576, filed 14 Sep. 2016.

### FIELD OF THE INVENTION

The invention relates to proliposomal powder dispersion formulations and oral dosage forms for the improved delivery of testosterone undeconoate.

### BACKGROUND

The goal of testosterone replacement therapy (TRT) is to restore plasma testosterone levels to a normal range and to alleviate symptoms suggestive of hormone deficiency, or for some individuals, a more masculine appearance and identity. This can be accomplished in a variety of ways, although a convenient form of TRT relies on oral administration of testosterone undecanoate (TU). However, the performance of currently marketed oral dosage forms for TU varies significantly, depending on when an individual ingests the dosage form, relative to meal times. See Yin et al. The inventions described below overcome longstanding diet-dependent obstacles to oral TU administration. The following description shows that TU can be incorporated into a dry, free-flowing powder that will form readily absorbable liposome-encapsulated TU. Moreover, because proliposomal formulation are dry powders, they, unlike liquid suspensions of liposomes, can be incorporated into oral dosage forms which are coated with a delayed release coating (e.g., an enteric coating) that will protect the formulation until it reaches the less hostile, aqueous environment of the small intestine, where hydration of the prolipomal powder dispersion can occur to cause the formation of liposomes that deliver TU to the intestinal epithelium.

### SUMMARY OF THE INVENTION

The invention relates to compositions of proliposomal formulations that contain a proliposomal powder dispersion of testosterone undecanoate (TU) and distearoyl phosphatidylcholine (DSPC). These powder dispersions can be incorporated into oral dosage forms which are used to deliver an effective dose of TU with minimal intererence from food effects, and therefore, are useful for treating diseases, disorders, or conditions characterized by testosterone deficiency.

The proliposomal powder dispersions of the invention are characterized by containing TU and DSPC in specified weight/weight (w/w) ratios that correlate with significant improvements in TU release and bioavailability. More specifically, the (TU) and (DSPC) are present in the dispersion in a w/w ratio of (a):(b), respectively, that ranges from (1.0:1.0) to (1.0:4.0).

The proliposomal powder dispersion of an oral dosage form of the invention contains a TU dosage amount equivalent to a therapeutic dose (human equivalent dose) of testosterone 60 to 729 mg per day, and is in the form of a capsule with a delayed release coating. Coated oral dosage forms of the invention can be used to bring the plasma concentration of testosterone of an individual suffering from low endogenous testosterone levels to a normal physiological concentration.

### BRIEF DESCRIPTION OF THE FIGURES

FIG. 1 shows dissolution data for the following TU formulations: TU1-044 (non-coated, non-capsulated, unformulated TU); TU1-076 (coated, capsulated, unformulated TU); TU1-040 (coated, capsulated TU:DSPC:Chol (1:0.9:0.1)); TU1-061c (coated, capsulated TU:DSPC:Chol:TPGS (1:0.9:0.1:0.05)); TU1-061a (coated, capsulated TU:DSPC:Chol:TPGS (1:0.9:0.1:0.2)); TU2-027 (coated, capsulated TU:DSPC (1:1)); TU2-028 (coated, capsulated TU:DSPC (1:2)); TU2-029 (coated, capsulated TU:DSPC (1:4)); and TU2-030 (coated, capsulated TU:90 HH (1:1)).

FIG. 2A shows plasma ‘T’ levels over a 24 hour time period in female beagle dogs following oral administration of testosterone (T) formulation TSX-002 coated, capsulated T:DSPC:Chol (1:0.9:0.1)) at days 1 and 7 of being places under fasted and fed conditions. T dosage=7.5 mg/kg/OD. Legend: Day 1 fasted, TSX-002—Solid black line and circle time point markers; Day 7 fasted TSX-002—Hashed black line and circle time point markers; Day 1 fed, TSX-002—Dotted black line and triangle time point markers; Day 1 fasted unformulated T—Solid grey line and triangle marking time points; Day 7 fasted unformulated T—Solid light grey line and square time point markers; Day 1 fed unformulated T—Solid black line and square time point markers; and Day 7 fed unformulated T—Solid grey line and crosshair time point markers.

FIG. 2B shows plasma ‘T’ levels over a 24 hour time period in female beagle dogs following oral administration of testosterone (T) formulation TSX-007 coated, capsulated T:DSPC:Chol:TPGS (1:0.9:0.1:0.2)) at days 1 and 7 of being places under fasted and fed conditions. T dosage=7.5 mg/kg/OD. Legend: Day 1 fasted, TSX-007—Solid black line and circle time point markers; Day 7 fasted TSX-007—Hashed black line and circle time point markers; Day 1 fed, TSX-007—Dotted black line and triangle time point markers; Day 1 fasted unformulated T—Solid grey line and triangle marking time points; Day 7 fasted unformulated T—Solid light grey line and square time point markers; Day 1 fed unformulated T—Solid black line and square time point markers; and Day 7 fed unformulated T—Solid grey line and crosshair time point markers.

FIG. 2C shows plasma ‘T’ levels over a 24 hour time period in female beagle dogs following oral administration of testosterone undecanoate (TU) formulation TSX-009 coated, capsulated (TU:DSPC:Chol:TPGS:MC (1.0:0.9:0.1:0.2:0.6)) at days 1 and 7 of being places under fasted and fed conditions. TU dosage=7.5 mg/kg/OD. Legend: Day 1 fasted, TSX-009—Solid black line and circle timepoint markers; Day 7 fasted TSX-009—Hashed black line and circle timepoint markers; Day 1 fed, TSX-009—Dotted black line and triangle timepoint markers; Day 1 fasted unformulated T—Solid grey line and triangle marking timepoints; Day 7 fasted unformulated T—Solid light grey line and square timepoint markers; Day 1 fed unformulated T—Solid black line and square timepoint markers; and Day 7 fed unformulated T—Solid grey line and crosshair timepoint markers.

FIG. 3A shows plasma ‘T’ levels at day 1 (n=6) and day 7 (n=6) of unformulated TU treatment in female beagle dogs following oral administration of 7.5 mg/kg/QD under fasted conditions.

FIG. 3B shows plasma ‘T’ levels in the responders, only, from the group represented in FIG. 3A, at day 1 (n=2) and day 7 (n=3).

FIG. 3C shows plasma ‘T’ levels at day 1 (n=6) and day 7 (n=6) of unformulated TU treatment in female beagle dogs following oral administration of 7.5 mg/kg/QD under fed conditions.

FIG. 3D shows plasma ‘T’ levels in the responders, only, from the group represented in FIG. 3C, day 1 (n=6) and day 7 (n=5).

FIG. 4A shows plasma ‘T’ levels over a 24 hour time period TU treatment with formulations TSX-010 (TU:Lipid, 1:1), TSX-011 (TU:Lipid, 1:2), and TSX-012 (TU:Lipid, 1:4) in female beagle dogs following oral administration of 1.875 mg/kg/QD under fasted conditions. (n=4 for each formulation)

FIG. 4B shows plasma ‘T’ levels in the responders, only, from the groups represented in FIG. 4A, (n=1 for TSX-010, n=2 for TSX-011, n=3 for TSX-012)

FIG. 5A shows plasma ‘T’ levels over a 24 hour time period following TU treatment with formulations TSX-010 (TU:Lipid, 1:1), TSX-011 (TU:Lipid, 1:2), and TSX-012 (TU:Lipid, 1:4) in female beagle dogs following oral administration of 3.75 mg/kg/QD under fasted conditions.

FIG. 5B shows plasma ‘T’ levels in the responders, only, from the groups represented in FIG. 5A, (n=1 for TSX-010, n=2 for TSX-011, n=4 for TSX-012)

FIG. 6A shows plasma ‘T’ levels over a 24 hour time period following TU treatment with formulations TSX-010 (TU:Lipid, 1:1), TSX-011 (TU:Lipid, 1:2), and TSX-012 (TU:Lipid, 1:4) in female beagle dogs following oral administration of 7.5 mg/kg/QD under fasted conditions.

FIG. 6B shows plasma ‘T’ levels in the responders, only, from the groups represented in FIG. 6A, (n=1 for TSX-010, n=3 for TSX-011, n=2 for TSX-012)

FIG. 7A shows plasma ‘T’ levels over a 24 hour time period TU treatment with formulations TSX-010 (TU:Lipid, 1:1), TSX-011 (TU:Lipid, 1:2), and TSX-012 (TU:Lipid, 1:4) in female beagle dogs following oral administration of 1.875 mg/kg/QD under fed conditions.

FIG. 7B shows plasma ‘T’ levels in the responders, only, from the groups represented in FIG. 7A, (n=3 for TSX-010, n=2 for TSX-011, n=2 for TSX-012)

FIG. 8A shows plasma ‘T’ levels over a 24 hour time period following TU treatment with formulations TSX-010 (TU:Lipid, 1:1), TSX-011 (TU:Lipid, 1:2), and TSX-012 (TU:Lipid, 1:4) in female beagle dogs following oral administration of 3.75 mg/kg/QD under fed conditions.

FIG. 8B shows plasma ‘T’ levels in the responders, only, from the groups represented in FIG. 8A, (n=3 for TSX-010, n=3 for TSX-011, n=4 for TSX-012)

FIG. 9A shows plasma ‘T’ levels over a 24 hour time period following TU treatment with formulations TSX-010 (TU:Lipid, 1:1), TSX-011 (TU:Lipid, 1:2), and TSX-012 (TU:Lipid, 1:4) in female beagle dogs following oral administration of 7.5 mg/kg/QD under fed conditions.

FIG. 9B shows plasma ‘T’ levels in the responders, only, from the groups represented in FIG. 9A, (n=4 for TSX-010, n=4 for TSX-011, n=4 for TSX-012)

FIG. 10A shows plasma ‘T’ levels over a 24 hour time period following TU treatment with 1.875, 3.75, and 7.5 mg/kg/QD formulation TSX-010 (TU:Lipid, 1:1) under fasted conditions. (n=4 for each dosage)

FIG. 10B shows plasma ‘T’ levels over a 24 hour time period following TU treatment with 1.875, 3.75, and 7.5 mg/kg/QD formulation TSX-010 (TU:Lipid, 1:1) under fed conditions. (n=4 for each dosage)

FIG. 11A shows plasma ‘T’ levels over a 24 hour time period following TU treatment with 1.875, 3.75, and 7.5 mg/kg/QD formulation TSX-011 (TU:Lipid, 1:2) under fasted conditions. (n=4 for each dosage)

FIG. 11B shows plasma ‘T’ levels over a 24 hour time period following TU treatment with 1.875, 3.75, and 7.5 mg/kg/QD formulation TSX-011 (TU:Lipid, 1:2) under fed conditions. (n=4 for each dosage)

FIG. 12A shows plasma ‘T’ levels over a 24 hour time period following TU treatment with 1.875, 3.75, and 7.5 mg/kg/QD formulation TSX-012 (TU:Lipid, 1:4) under fasted conditions. (n=4 for each dosage)

FIG. 12B shows plasma ‘T’ levels over a 24 hour time period following TU treatment with 1.875, 3.75, and 7.5 mg/kg/QD formulation TSX-012 (TU:Lipid, 1:4) under fed conditions. (n=4 for each dosage)

FIG. 13A shows plasma ‘T’ levels over a 24 hour time period following TU treatment at 7.5 mg/kg/QD & BID formulation TSX-011 (TU:Lipid, 1:2) under fasted conditions. (n=6 for each dosage)

FIG. 13B shows plasma ‘T’ levels over a 24 hour time period following TU treatment at 7.5 mg/kg/QD & BID formulation TSX-011 (TU:Lipid, 1:2) under fed conditions. (n=6 for each dosage)

FIG. 14A shows plasma ‘T’ levels over a 24 hour time period following TU treatment at 3.75 mg/kg/BID formulation TSX-011 (TU:Lipid, 1:2) under fed conditions on day 1 and day 7. (n=6 for each dosage)

FIG. 14B shows plasma ‘T’ levels over a 24 hour time period following TU treatment at 7.5 mg/kg/BID formulation TSX-011 (TU:Lipid, 1:2) under fed conditions on day 7. (n=6 for each dosage)

FIG. 14C shows plasma ‘T’ levels over a 24 hour time period following TU treatment at 11.25 mg/kg/BID formulation TSX-011 (TU:Lipid, 1:2) under fed conditions on day 7. (n=6 for each dosage)

FIG. 14D shows plasma ‘T’ levels over a 24 hour time period following TU treatment at 3.75, 7.5, 11.25 mg/kg/BID formulation TSX-011 (TU:Lipid, 1:2) under fed conditions on day 1 & day 7. (n=6 for each dosage)

FIG. 14E Dose proportionality curve following TU treatment at 3.75, 7.5, 11.25 mg/kg/BID formulation TSX-011 (TU:Lipid, 1:2) under fed conditions on day 7. (n=6 for each dosage)

### DETAILED DESCRIPTION

The invention relates to compositions of proliposomal formulations of testosterone undecanoate (TU), and oral dosage forms that contain proliposomal formulations of TU, which are used to deliver an effective dose of TU with minimal food effects. The invention also relates to methods for preparing proliposomal formulations and dosage forms of the invention, and methods and uses of the formulations and dosage forms of the invention for treating diseases, disorders, or conditions characterized by testosterone deficiency.

### Composition of Proliposomal Powder Dispersions

A proliposomal formulation of the invention at least contains a proliposomal powder dispersion of TU and distearoyl phosphatidylcholine (DSPC), which are combined in a specified weight/weight (w/w) ratio that correlates with significant improvements in TU release and bioavailability. More specifically, the (TU) and (DSPC) are present in the dispersion in a w/w ratio of (a):(b), respectively, that ranges from (1.0:1.0) to (1.0:4.0). Therefore, (TU) and (DSPC) are present in a proliposomal powder dispersion of the invention in a w/w ratio of (a):(b) that is (1.0:1.10), (1.0:1.20), (1.0:1.30), (1.0:1.40), (1.0:1.50), (1.0:1.60), (1.0:1.70), (1.0:1.80), (1.0:1.90), (1.0:2.00), (1.0:2.10), (1.0:2.20), (1.0:2.30), (1.0:2.40), (1.0:2.50), (1.0:2.60), (1.0:2.70), (1.0:2.80), (1.0:2.90), (1.0:3.00), (1.0:3.10), (1.0:3.20), (1.0:3.30), (1.0:3.40), (1.0:3.50), (1.0:3.60), (1.0:3.70), (1.0:3.80), (1.0:3.90), (1.0:4.0), or any w/w ratio therein. A preferred proliposomal powder dispersion of the invention contains TU and DSPC in a w/w ratio, (a):(b), of (1.0:2.0).

A proliposomal powder dispersion of the invention can also consist essentially of (TU) and (DSPC) in a w/w ratio of (a):(b) that is (1.0:1.10), (1.0:1.20), (1.0:1.30), (1.0:1.40), (1.0:1.50), (1.0:1.60), (1.0:1.70), (1.0:1.80), (1.0:1.90), (1.0:2.00), (1.0:2.10), (1.0:2.20), (1.0:2.30), (1.0:2.40), (1.0:2.50), (1.0:2.60), (1.0:2.70), (1.0:2.80), (1.0:2.90), (1.0:3.00), (1.0:3.10), (1.0:3.20), (1.0:3.30), (1.0:3.40), (1.0:3.50), (1.0:3.60), (1.0:3.70), (1.0:3.80), (1.0:3.90), (1.0:4.0), or any w/w ratio therein. A preferred proliposomal powder dispersion of the invention consists essentially of TU and DSPC in a w/w ratio, (a):(b), of (1.0:2.0).

A proliposomal powder dispersion of the invention can also consist of (TU) and (DSPC) in a w/w ratio of (a):(b) that is (1.0:1.10), (1.0:1.20), (1.0:1.30), (1.0:1.40), (1.0:1.50), (1.0:1.60), (1.0:1.70), (1.0:1.80), (1.0:1.90), (1.0:2.00), (1.0:2.10), (1.0:2.20), (1.0:2.30), (1.0:2.40), (1.0:2.50), (1.0:2.60), (1.0:2.70), (1.0:2.80), (1.0:2.90), (1.0:3.00), (1.0:3.10), (1.0:3.20), (1.0:3.30), (1.0:3.40), (1.0:3.50), (1.0:3.60), (1.0:3.70), (1.0:3.80), (1.0:3.90), (1.0:4.0), or any w/w ratio therein. A preferred proliposomal powder dispersion of the invention consists of TU and DSPC in a w/w ratio, (a):(b), of (1.0:2.0).

### Preparation of Proliposomal Powder Dispersions

A proliposomal powder dispersion of the invention can be prepared by dissolving TU in a solvent. Heat, (e.g., 45-55° C.) can optionally be applied during dissolution. The solvent is any solvent in which TU dissolves, but is preferably a water-miscible solvent such as ethanol; however, the solvent should generally not contain 10% or more of water (vol/vol). Other exemplary solvents include methanol, chloroform, dichloromethane, acetone, isopropyl alcohol, and diethyl ether. Upon dissolution of TU (i.e. the solution becomes clear), DSPC is also dissolved into the TU solution until the solution again becomes clear. The solvent is removed by any suitable technique, such as, by evaporation, by placing the solution under vacuum, by spray-drying, or by use of a drying gas, and the like. The solvent removal process continues until a dry mass of the TU and DSPC dispersion forms. The average particle size of resulting powder dispersion can be reduced by grinding, passing the powder through screens, or by any other suitable technique. For example, the particles within a proliposomal powder dispersion can have powder size ranging from about 10 to 200 mesh, 20 to 120 mesh or 40 to 60 or 60 to 80 mesh. If desired, the proliposomal powder dispersion can undergo further drying to remove or reduce the amount of any residual solvent still present in the powder. Such a further drying step is performed by using one or more of the drying techniques discussed above or by other suitable drying technique.

### Oral Dosage Forms

An oral dosage form of the invention contains a proliposomal powder of the invention, which contains a therapeutic dose of TU 95 to 1152 mg per day. Generally, a therapeutic dosage amount of TU corresponds to an equivalent amount of testosterone by a factor of 1.58 (i.e., 1 mg of T==1.58 mg TU).

Such oral dosage forms, can also contain one or more pharmaceutically acceptable excipients in addition to a proliposomal powder. Generally, an excipient or excipients, in an oral dosage form of the invention are added externally to the proliposomal powder dispersion. In other words, excipients are admixed with a dry proliposomal powder dispersion containing TU and DSPC. For example, an oral dosage form of the invention can contain a proliposomal powder dispersion of the invention admixed with microcrystalline cellulose, or sodium starch glycolate, or both.

Further to the foregoing examples of microcrystalline cellulose and sodium starch glycolate, other exemplary pharmaceutically acceptable excipients for oral dosage forms of the invention include: (a) fillers or extenders, such as starches, lactose (e.g., lactose monohydrate), sucrose, glucose, mannitol, and silicic acid; (b) binders, such as cellulose derivatives like microcrystalline cellulose (e.g., the various Avicel® PH products like Avicel® PH-101 and PH-102, and Prosolv® products like Prosolv® SMCC 90 and 90 HD), starch, aliginates, gelatin, polyvinylpyrrolidone, sucrose, and gum acacia; (c) humectants, such as glycerol; (d) disintegrating agents, such as agar-agar, calcium carbonate, potato or tapioca starch, sodium starch glycolate (e.g., Explotab® disintegrant), alginic acid, croscarmellose sodium, complex silicates, and sodium carbonate; (e) solution retarders, such as and paraffin; (f) absorption accelerators, such as quaternary ammonium compounds; (g) wetting agents, such as, for example, cetyl alcohol, and glycerol monostearate, and magnesium stearate; (h) adsorbents, such as kaolin and bentonite; (i) lubricants, such as talc, calcium stearate, magnesium stearate, solid polyethylene glycols, and sodium lauryl sulfate (SLS); (j) plasticizers; and (k) dispersants, including mannitol (e.g., Pearlitol® SD 2000).

The w/w ratio of an excipient to the proliposomal powder dispersion component of an oral dosage form of the invention may be, but is not necessarily, critical to its desired TU release characteristics. More specifically, pharmacokinetic (PK) parameters, such as blood plasma testosterone concentration, area under the curve (AUC), maximum plasma concentration (Cmax), and amount of time taken to reach the maximum concentration (Tmax) values may correlate to certain w/w ratios of proliposomal powder dispersion to excipients. For example, the w/w ratio of a proliposomal powder dispersion to microcrystalline cellulose in an oral dosage form of the invention can be (1.0:1.0), (1.0:1.01), (1.0:1.02), (1.0:1.03), (1.0:1.04), (1.0:1.05), (1.0:1.06), (1.0:1.07), (1.0:1.08), (1.0:1.09), or (1.0:1.10), (1.0:1.5), (1.0:2.0), (1.0:1.3.0), (1.0:4.0), or any ratio therein. A preferred oral dosage form contains a proliposomal powder dispersion of TU and DSPC in a w/w ratio, (a):(b), of (1.0:2.0) in combination with microcrystalline cellulose in a w/w ratio of (1.0:1.06).

In another example of a correlatation of desirable PK characteristics of an oral dosage form of the invention to certain w/w ratios of proliposomal powder dispersion to excipients, the w/w ratio of a proliposomal powder dispersion to sodium starch glycolate (SSG) in an oral dosage form of the invention can be (1.0:0.050), (1.0:0.051), (1.0:0.052), (1.0:0.053), (1.0:0.054), (1.0:0.055), (1.0:0.056), (1.0:0.057), (1.0:0.058), (1.0:0.059), (1.0:0.060), (1.0:0.061), (1.0:0.062), (1.0:0.063), (1.0:0.064), (1.0:0.065), (1.0:0.066), (1.0:0.067), (1.0:0.068), (1.0:0.069), (1.0:0.070), (1.0:0.071), (1.0:0.072), (1.0:0.073), (1.0:0.074), (1.0:0.075), (1.0:0.076), (1.0:0.077), (1.0:0.078), (1.0:0.079), (1.0:0.080), (1.0:0.0.09), (1.0:0.10), (1.0:0.20) or any ratio therein. A preferred oral dosage form contains a proliposomal powder dispersion of TU and DSPC in a w/w ratio, (a):(b), of (1.0:2.0) in combination with SSG in a w/w ratio of (1.0:0.064).

Another preferred oral dosage form of the invention contains a proliposomal powder dispersion of TU and DSPC in a w/w ratio, (a):(b), of (1.0:2.0) in combination with microcrystalline cellulose and SSG in w/w ratios of dispersion:microcrystalline cellulose:SSG of 1.0:1.06:0.064. Yet another preferred oral dosage form of the invention consists, or optionally, consists essentially of, a proliposomal powder dispersion of TU and DSPC in a w/w ratio, (a):(b), of (1.0:2.0) in combination with microcrystalline cellulose and SSG in w/w ratios of dispersion:microcrystalline cellulose:SSG of 1.0:1.06:0.064.

An oral dosage form of the invention contains a therapeutic dose, or partial therapeutic dose, of TU, which for an adult human, is from 95.9 to 1,580 mg/day, the equivalent of 60.75 to 1000 mg of testosterone per day. For example, a preferred oral dosage form of the invention can contain about (i.e., within 10% of) 95 mg, 120 mg, 190 mg, 380 mg TU, or 760 mg of TU.

An oral dosage form of the invention is typically a capsule. More specifically, a capsule dosage form of the invention can be soft or hard capsule, and is generally made from animal-derived gelatin or plant-derived hydroxypropyl methylcellulose (HPMC). The size of a capsule for an oral dosage form of the invention can be any size that is sufficient to contain its proliposomal powder dispersion and excipient components. For example, the capsule can be a size 5, 4, 3, 2, 1, 0, 0E, 00, 000, 13, 12, 12el, 11, 10, 7, or Su07. Capsules are filled using any suitable techniques.

Filled capsules can be coated with a delayed release coating, also referred to as an enteric coating. A delayed release coating protects an oral dosage form of the invention from the harsh, acidic environment of the stomach, so that the release of the proliposomal powder dispersion can be delayed until the dosage form reaches the small intestine. Upon contact with small intestinal fluid, the proliposomal powder dispersion is hydrated, leading to the formation of liposomes and uptake of the TU through the small intestine epithelium or lymphatic system, or both. Any coatings of oral dosage forms of the invention are applied to a sufficient thickness such that the entire coating does not dissolve in the gastrointestinal fluids at pH below about 5.

A delayed release coating typically includes a polymer, such as an aqueous dispersion of anionic polymers with methacrylic acid as a functional group like the product sold as Eudragit® L30D-55 (Evonik Industries). A delayed release coating can also optionally include a plasticizer, such as triethyl citrate, an anti-tacking agent, such as talc, and a diluent, such as water. For example, a coating composition used to coat and oral dosage form of the invention can contain about 42% (wt %) of an aqueous dispersion of anionic polymers with methacrylic acid as a functional group; about 1.25 wt % of a plasticizer; about 6.25 wt % of an anti-tacking agent; and about 51 wt % of a diluent. Another example of a coating composition for an oral dosage form of the invention, particularly when a large-scale preparation is preferred, an appropriate amount of an anionic copolymer based on methacrylic acid and ethyl acrylate, such as Eudragit® L100-55, is used in place of Eudragit® L30D-55. Conventional coating techniques such as spray or pan coating are employed to apply coatings. For example, a coating composition can be applied to capsules of the invention by using a Procept® coating machine and Caleva® mini coater air suspension coating machine to coat the capsules until they experience a 10% to 18% weight gain.

### Testosterone Replacement Therapy

The proliposomal powder dispersions and oral dosage forms of the invention can be used for testosterone replacement therapy (TRT). Low endogenous testosterone is another term used to describe a sub-physiological testosterone level, which is generally considered to be plasma testosterone concentration of less than 300 ng/dL. Low endogenous testosterone levels can result from consequences of injury, infection, loss of testicles, chemotherapy, radiation treatment, genetic abnormalities, hemochromatosis, dysfunction of the pituitary gland, inflammatory disease, medication side effect, chronic kidney failure, liver cirrhosis, stress, alcoholism, obesity, Kallman's syndrome, idiopathic gonadotropin deficiency, Klinefelter's syndrome, pituitary hypothalamus injury due to tumours, osteoporosis, diabetes mellitus, chronic heart failure, chemotherapy, hemochromatosis, cirrhosis, renal failure, AIDS, sarcoidosis, Kallman's Syndrome, androgen receptor defects, 5-alpha reductase deficiency, myotonic dystrophy, cryptorchidism, mumps orchitis, aging, fertile eunuch syndrome, and pituitary disorders.

Another condition that can be treated with proliposomal powder formulations or oral dosage forms of the invention is male hypogonadism, or testosterone deficiency syndrome (TDS), which results from a failure of the testes to produce adequate androgen. Patients have low circulating testosterone in combination with clinical symptoms such as fatigue, erectile dysfunction, and body composition changes. The cause may be primary (genetic anomaly, Klinefelter's syndrome) or secondary (defect in hypothalamus or pituitary), but often presents with the same symptomatology. In the older patient, androgen deficiency of the aging male (ADAM) is an important cause of secondary hypogonadism because testosterone levels decline progressively after age 40. Hypogonadal patients have alterations not only in sexual function and body composition, but also in cognition and metabolism. Regardless of etiology, hypogonadal patients who are both symptomatic and who have clinically significant alterations in laboratory values are candidates for treatment.

The administration of oral dosage forms of the invention can be used to raise an individual's plasma concentration of testosterone to a range of 300 ng/dL to 1050 ng/dL (including, 400 ng/dL to 950 ng/dL, 500 ng/dL to 950 ng/dL, and 600 ng/dL to 950 ng/dL) within five hours after administration under fasting or fed conditions. Daily TU dosage amounts administered in oral dosage forms of the invention, employed for adult human TRT can be from 96 to 1,580 mg/day, which is the equivalent of about 60.75 to 1000 mg of testosterone/day. Preferred daily TU dosage amounts administered in oral dosage forms of the invention, employed for adult human TRT are about 95 mg/60 kg body weight, about 192 mg/60 kg body weight, about 384 mg/60 kg body weight, about 768 mg/60 kg body weight, or about 1,152 mg/60 kg body weight.

In certain instances, it is appropriate to administer an oral dosage form of the invention in with another therapeutic agent. When such combinational therapy is employed, the other therapeutic agent can be separately administered, and administered by a different route. The other therapeutic agent can be administered concurrently (e.g., simultaneously, essentially simultaneously or within the same treatment protocol) or sequentially, depending upon the nature of the disease, the condition of the patient, and the actual choice of compounds used.

### Kits/Articles of Manufacture

Proliposomal powder dispersions and oral dosage forms of the invention can be included as part of a kit or with an article of manufacture. A kit can include a carrier, package, or container that is optionally compartmentalized to receive one or more doses of TU contained within a proliposomal powder dispersion or oral dosage forms of the invention. The kits provided herein contain packaging materials. Examples of pharmaceutical packaging materials include strip packs, blister packs, bottles, tubes, bags, containers, bottles, and any packaging material suitable for a selected formulation and intended mode of administration and treatment.

### EXAMPLES

### Example 1

Testosterone Undecanoate uncoated, no-lipid control formulation TU1-044. To prepare TU1-044, 95 mg of testosterone undecanoate (TU), purchased from Pfizer Inc., Kalamazoo, Mich., weighed and manually filled into uncoated size 1 Vcaps® Plus capsules.

### Example 2

Testosterone Undecanoate enteric-coated, no-lipid control formulation TU1-076. To prepare TU1-076, 95 mg of testosterone undecanoate (TU), purchased from Pfizer Inc., Kalamazoo, Mich., weighed and manually filled into uncoated size 1 Vcaps® Plus capsules. The filled capsules were coated with methacrylic add copolymer NF, type C (Eudragit® L 30D-55). Vcaps® Plus capsules contain United States Pharmacopeia (USP)-grade Hydroxypropyl Methylcellulose and water.

### Example 3

Testosterone Undecanoate+DSPC+cholesterol (1.0:0.9:0.1) formulation TU1-040. To prepare TU1-040, TU (3.95 g) was dissolved in 19 mL EtOH, at 45-55° C., and mixed until a clear solution formed. Distearoylphosphatidylcholine (DSPC) (3.55 g) and cholesterol (0.395 g) were added to the drug solution, and the mixture continued to mix at 45-55° C., until a clear solution formed. Mixing and heating continued under vacuum until a dried mass formed. The dried mass was passed through a sieve No 60. The dried and screened powder was filled into uncoated Size “0” Vcaps® Plus capsules.

### Example 4

Testosterone Undecanoate+DSPC+cholesterol+TPGS (1.0:0.9:0.1:0.05) formulation TU1-061c. To prepare TU1-061c, TU (1.9 g) was dissolved in 6.75 mL EtOH, at 45-55° C., and mixed until a clear solution formed. DSPC (1.710 g) and cholesterol (0.190 g) were added to the drug solution, and mixing was continued at 45-55° C., until a clear solution formed. Vitamin E TPGS (95 mg) was dispensed into a separate bowl, and dissolved by mixing it in EtOH (approximately 0.3 mL), based on the ratio of (1.2 g of TPGS/4 mL of EtOH). Microcrystalline cellulose (0.190 mg) (Avicel® PH 102) and 0.2 mL Ethanol were added to the TPGS solution, and mixed to form a slurry. The TPGS/microcellulose slurry was added to the TU/DSPC/Chol solution, and the combination was mixed at 45-55° C. under a vacuum until the whole slurry became an agglomerate mass or several large masses, which were then broken down into smaller agglomerates and subjected to continued drying under a vacuum. The dried mass was removed, and passed through a mill fitted with a Sieve No. 60 screen. Lumps of the dried mass that were hard to pass through the screen, were passed through a bigger screen before passing it through the smaller screen. The milled dry mass was filled into Size “00” Vcaps® Plus capsules.

### Example 5

Testosterone Undecanoate+DSPC+cholesterol+TPGS (1.0:0.9:0.1:0.2) formulation TU1-061a. To prepare TU1-061a, TU (1.9 g) was dissolved in 6.75 ml EtOH, at 45-55° C., and mixed until a clear solution formed. DSPC (1.710 g) and cholesterol (0.190 g) were added to the drug solution, and mixing continued at 45-55° C., until a clear solution formed. Vitamin E TPGS (0.380 g) was dispensed into a separate bowl, and dissolved into in EtOH (1.26 mL), based on the ratio of (1.2 g of TPGS/4 mL of EtOH). Microcrystalline cellulose (0.760 g) (Avicel® PH 102) and 0.8 mL EtOH were added to form a slurry. The TPGS/microcellulose slurry was added to the TU/DSPC/Chol solution, and the combination was mixed at 45-55° C. under a vacuum until the slurry became an agglomerate mass, or several large masses, which were then broken down into smaller agglomerates and subjected to continued drying under vacuum. The dried mass was removed, and passed through a mill fitted with a Sieve No. 60 screen. Lumps of the dried mass that were hard to pass through the screen, were passed through a bigger screen before passing it through the smaller screen. The milled dry mass, was filled into Size “00” Vcaps® Plus capsules.

### Example 6

Testosterone Undecanoate+DSPC (1.0:1.0) formulation TU1-027. To prepare TU1-027, TU (11.875 g) was dissolved in 40 ml EtOH, at 45-55° C., and mixed until a clear solution formed. DSPC (11.875 g) was added to the drug solution, and the mixture continued to mix at 45-55° C., until a clear solution formed. Mixing and heating continued under a vacuum until a dry mass was formed which, was then milled and screened through a sieve No 60 to obtained dry powder. Microcrystalline cellulose (71.80 g) (Avicel® PH 102) and sodium starch glycolate (2.9 g) (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55.

### Example 7

Testosterone Undecanoate+DSPC (1.0:2.0) formulation TU1-028. To prepare TU1-028, TU (11.875 g) was dissolved in 40 mL EtOH, at 45-55° C., and mixed until a clear solution formed. DSPC (23.75 g) was added to the drug solution, and the mixture continued to mix at 45-55° C., until a clear solution formed. Mixing and heating continued under a vacuum until a dry mass was formed which, was then milled and screened through a sieve No 60 to obtained dry powder. Microcrystalline cellulose (63.38 g) (Avicel® PH 102) and sodium starchglycolate (3.01 g) (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55.

### Example 8

Testosterone Undecanoate+DSPC (1.0:4.0) formulation TU1-029. To prepare TU1-029, TU (11.875 g) was dissolved in 40 mL EtOH, at 45-55° C., and mixed until a clear solution formed. DSPC (47.5 g) was added to the drug solution, and the mixture continued to mix at 45-55° C., until a clear solution formed. Mixing and heating continued under a vacuum until a dry mass was formed which, was then milled and screened through a sieve No 60 to obtain a dry powder. Microcrystalline cellulose (37.80 g) (Avicel® PH 102) and sodium starchglycolate (2.96 g) of (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55.

### Example 9

Testosterone Undecanoate+90 H (1.0:1.0) formulation TU1-030. To prepare TU1-030, TU (23.8 g) was dissolved in 40 mL EtOH, at 45-55° C., and mixed until a clear solution formed. Hydrogenated phosphatidylcholine 90 H (23.8 g) (purchased from Lipoid, LLC) was added to the drug solution, and the mixture continued to mix at 45-55° C., until a clear solution formed. Mixing and heating continued under vacuum until a dry mass was formed which was milled and screened through a sieve No 60 to obtain dry powder mass. Microcrystalline cellulose (66.82 g) (Avicel® PH 102), and sodium starch glycolate (2.76 g) (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55.

### Example 10

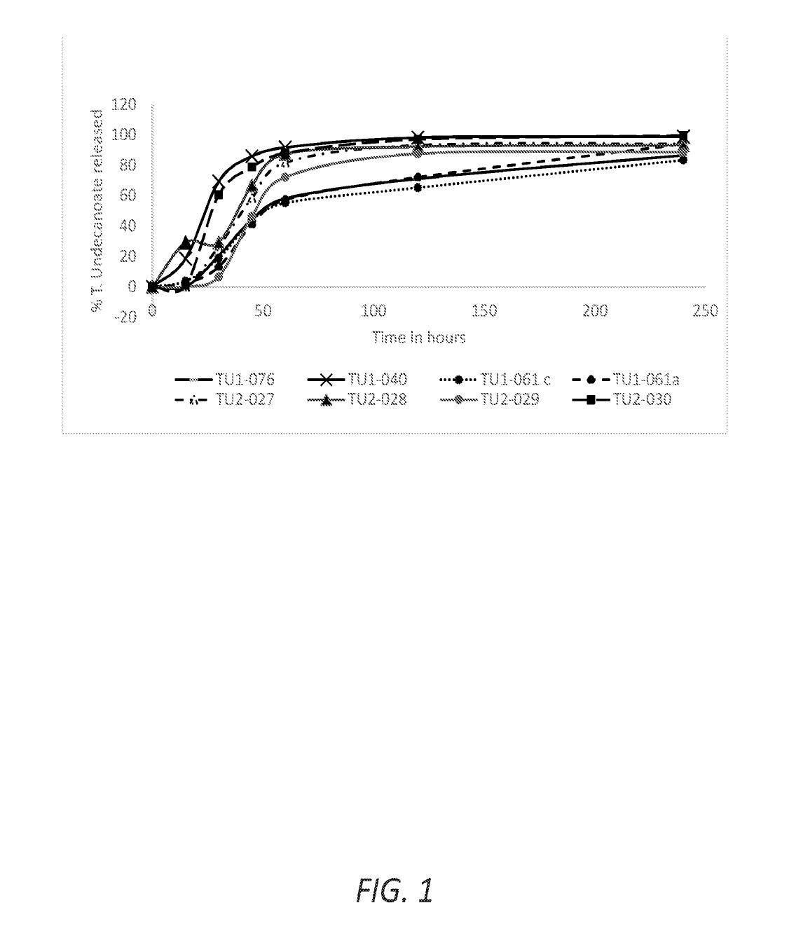

Dissolution of proliposomal TU formulations in different media. Dissolution studies were conducted for the control and proliposomal formulations described in Examples 1-9. With exception of the pure TU control formulation, dissolution data for each formulation was obtained by adding a capsule form of a formulation, containing 100 mg of TU to 750 mL of dissolution medium. These data are summarized in Table 1 and FIG. 1.

The dissolution method involved two stages of testing, the Acid stage and the Buffer stage. In the Acid stage, the dissolution was carried out in 750 mL of 0.1N HCl, and maintained at 37±0.5° C. for two hours. After two hours, a sample aliquot was withdrawn to be used in the buffer stage. The capsules were removed from the dissolution apparatus after 2 h of dissolution in 0.1N HCl To the same acid media, 250 ml of 0.2M Tribasic sodium phosphate with containing 1% w/v SLS was added (SLS was not included in dissolution medium for TU1-044). The final concentration of SLS in the combined media was 0.25% w/v. The pH of the media was adjusted 6.80 with 2N HCl or 2N NaOH. The dissolution data for control formulation TU1-044 did not contain SLS. The dissolution study was run for four hours in buffer stage, and sample aliquots were withdrawn at regular time intervals. The samples were analyzed using a suitable analytical technique.

A HPLC method was used for the analysis of dissolution samples. HPLC analysis was carried out using a gradient method. The mobile phase consisted of water and acetonitrile as follows: (90% water+10% acetonitrile) at 0 minutes; (4% water+96% acetonitrile) at 2 minutes; and (4% water+96% acetonitrile) at 15 minutes. Separation was achieved on a C18; 150×4.6 mm (5 μm) (Ace) column. The mobile phase flow rate was set at 1.4 mL/min. while the column temperature was maintained at 40° C. The total run time was 15 minutes with injection volume of 35 μl. The testosterone was detected using a UV detector at absorbance maxima of 243 nm. The retention time of testosterone was found to be around 10 minutes. The method was able to resolve testosterone undecanoate and all other excipients.

TABLE 1

| | | % TU dose released (Time in Minutes) | | | | | |
|---|---|---|---|---|---|---|---|
| Formulation | Composition | 15 | 30 | 45 | 60 | 120 | 240 |
| TU1-044 | Unformulated TU (95 mg) | 0.00 | 0.00 | 0.00 | 0.00 | 0.00 | 0.00 |
| TU1-076 | Enteric coated caps of | 0.00 | 20.51 | 44.29 | 58.08 | 71.15 | 86.54 |
| | unformulated TU (95 mg) | | | | | | |
| TU1-040 | T:DSPC:Chol (1:0.9:0.1) | 18.46 | 69.32 | 85.93 | 91.7 | 98.26 | 98.84 |
| TU1-061 c | TU:DSPC:Chol | 3.71 | 18.73 | 43.69 | 55.18 | 65.25 | 83.41 |
| | (1:0.9:0.1) + | | | | | | |
| | TPGS (5% weight of TU) | | | | | | |
| TU1-061a | TU:DSPC:Chol | 2.21 | 13.14 | 41.25 | 57.27 | 72.10 | 93.96 |
| | (1:0.9:0.1) + | | | | | | |
| | TPGS (20% weight of TU) | | | | | | |
| TU2-027 | TU:DSPC (1:1) | 0.00 | 26.61 | 58.43 | 81.78 | 93.27 | 94.35 |
| TU2-028 | TU:DSPC (1:2) | 29.20 | 29.20 | 66.78 | 87.34 | 92.26 | 93.19 |
| TU2-029 | TU:DSPC (1:4) | 0.00 | 6.54 | 46.12 | 72.16 | 87.64 | 88.66 |
| TU2-030 | TU:90 H H (1:1) | 0.97 | 60.59 | 78.82 | 88.05 | 96.99 | 99.56 |

### Example 11

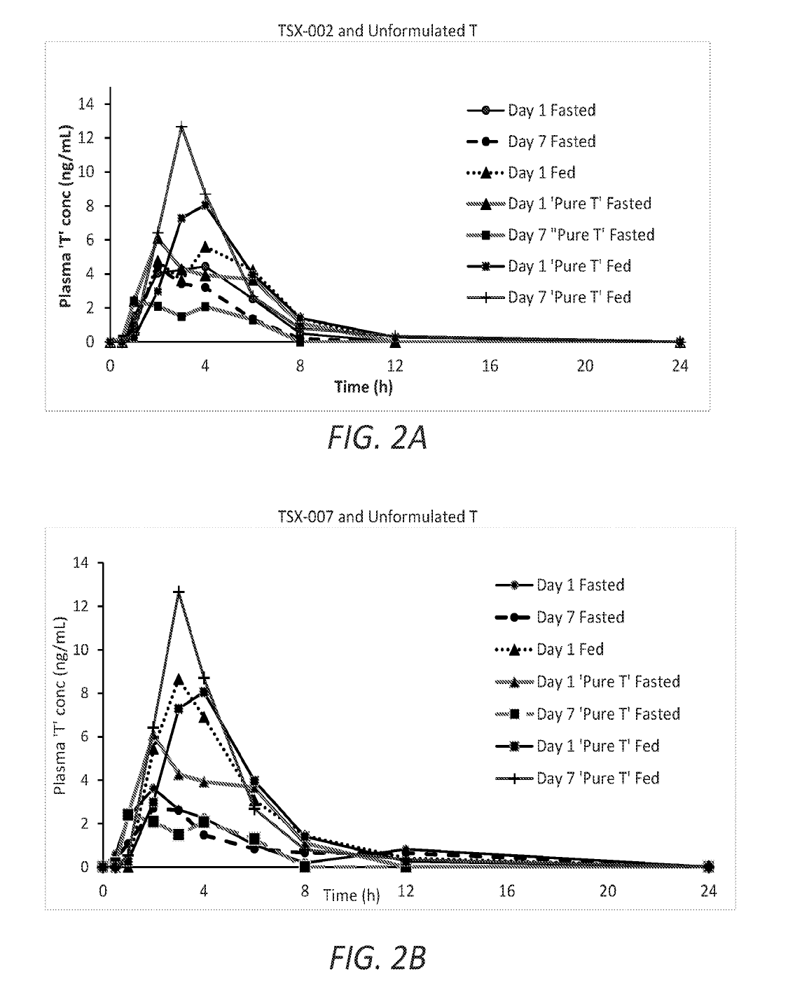

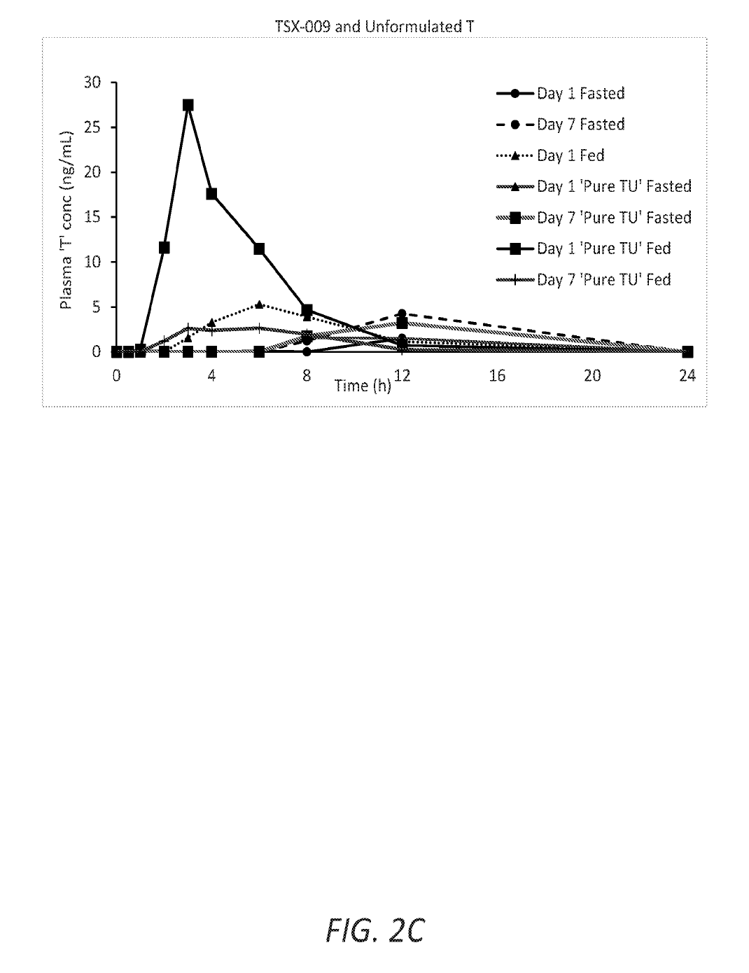

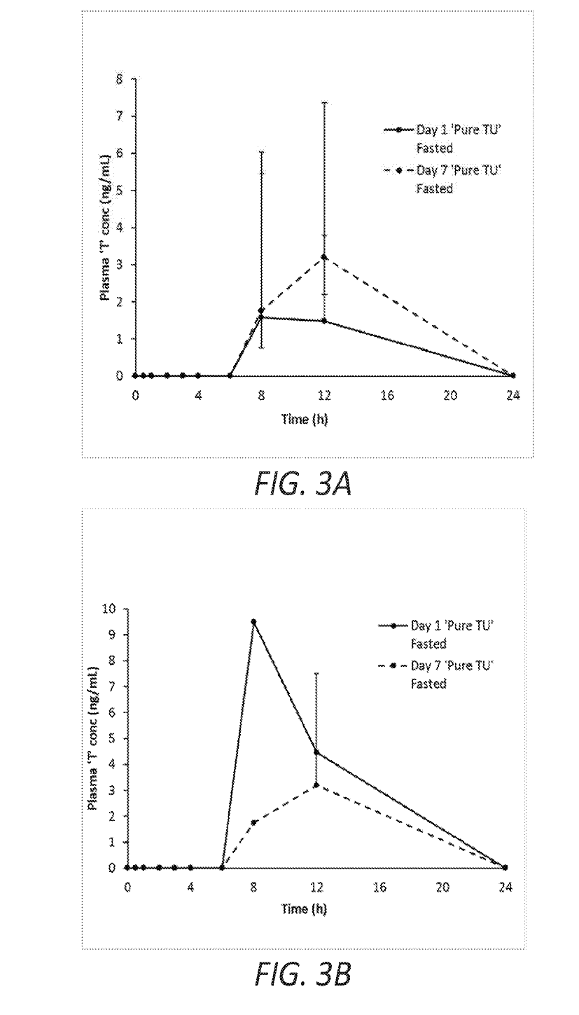

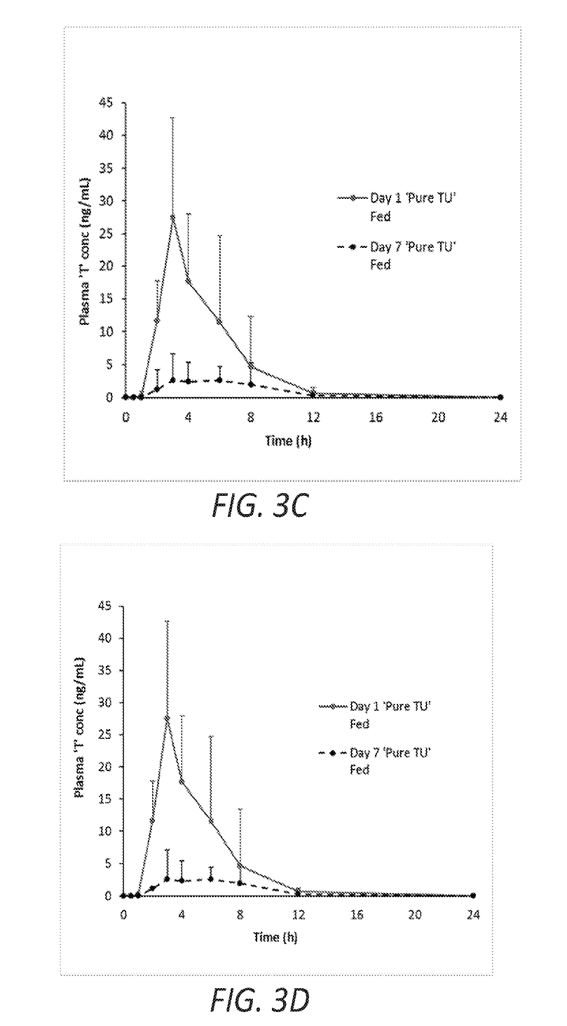

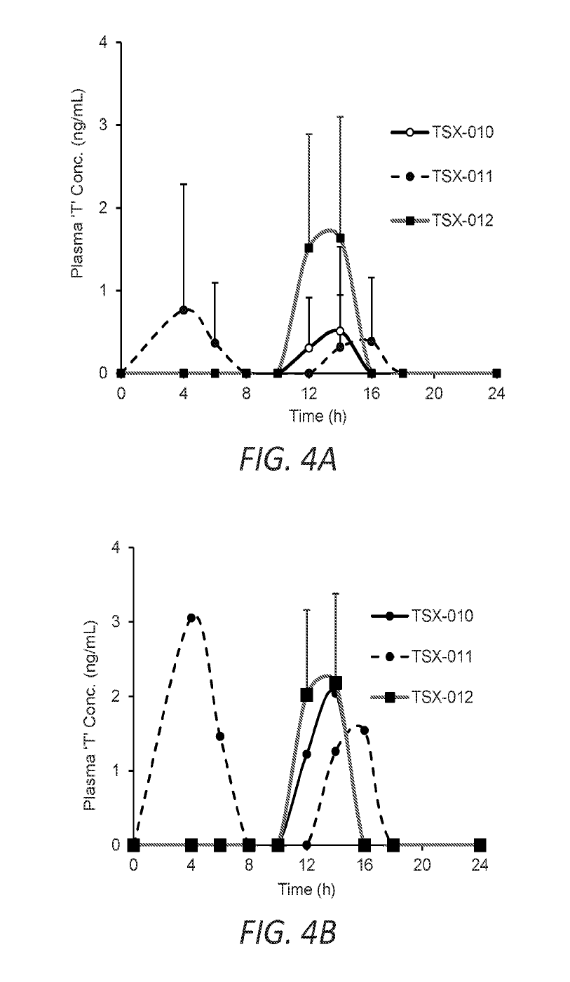

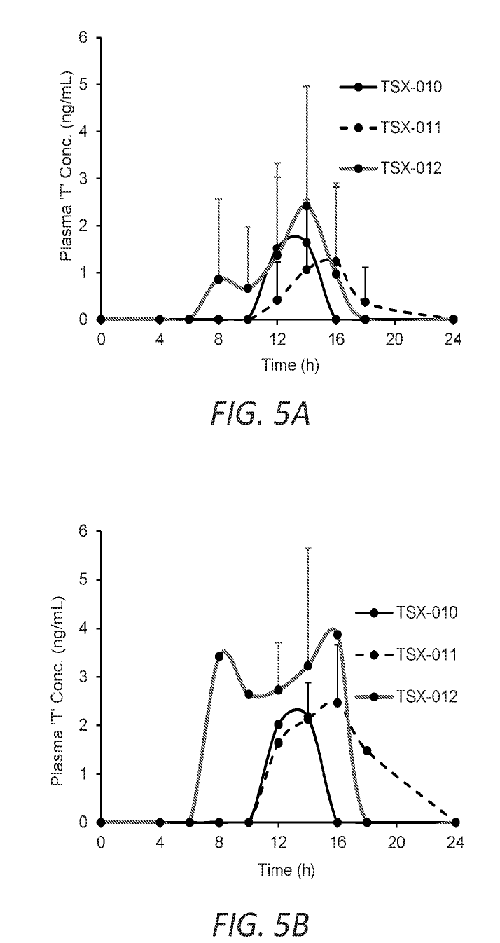

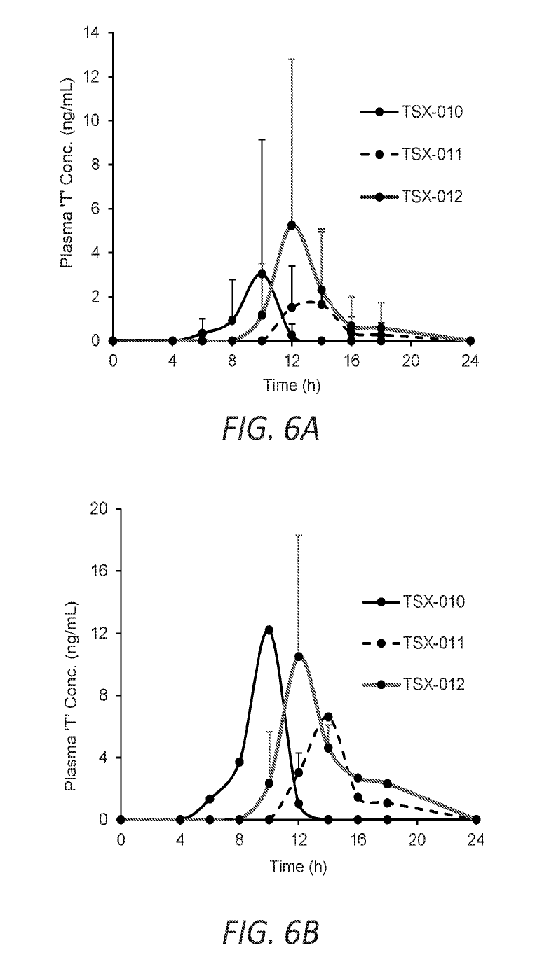

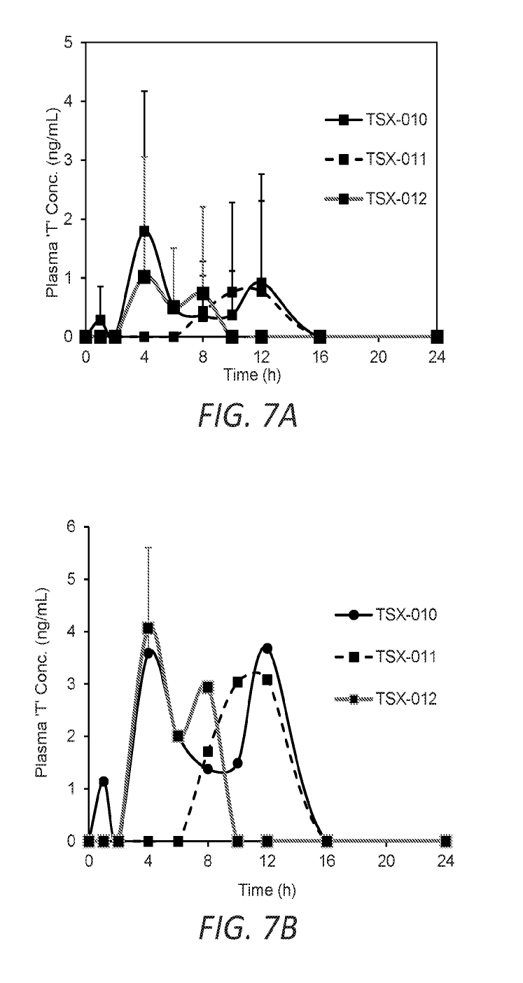

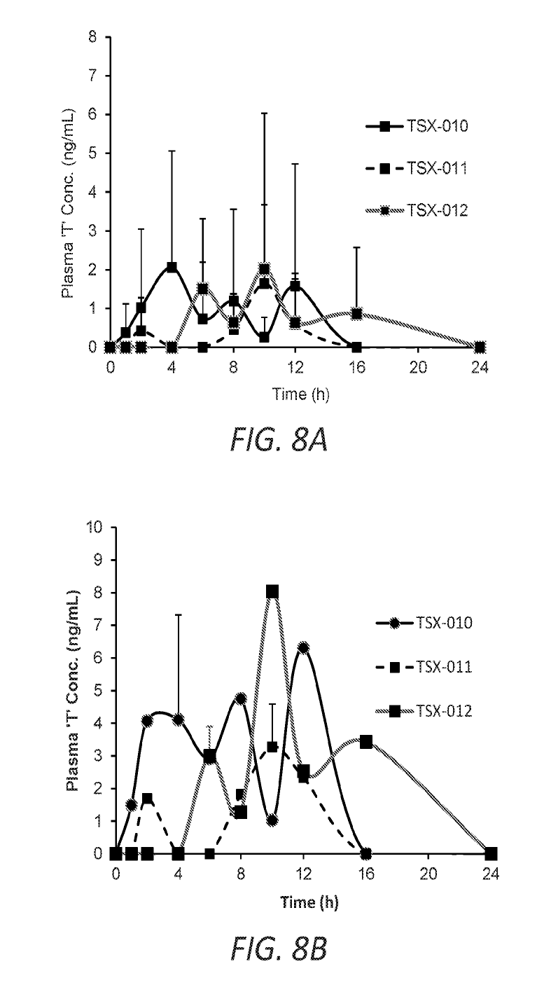

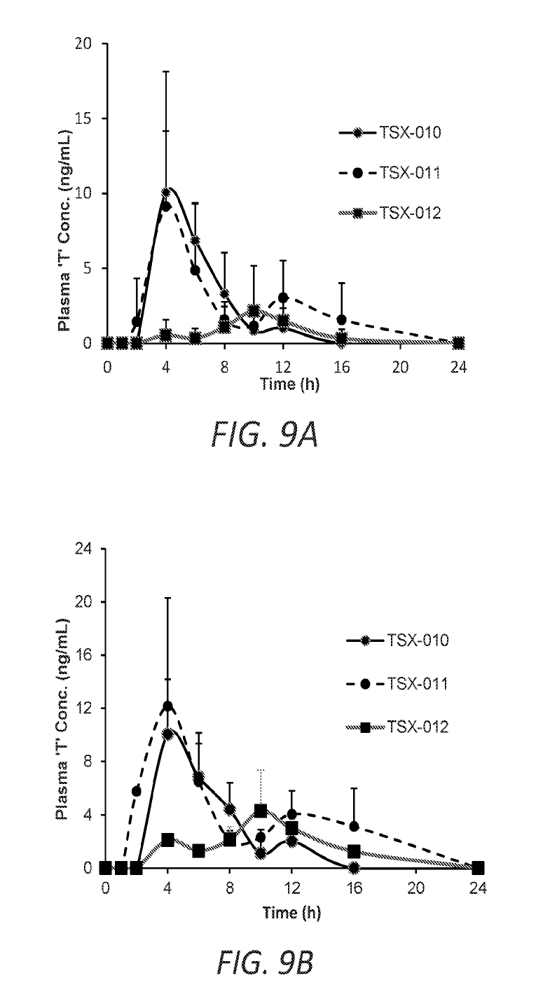

In vivo pK data TSX-002 (native T:Lipid), TSX-007 (native T:Lipid:TPGS (20% w/w of T)), and TSX-009 (TU:Lipid:TPGS (20% w/w of T)). TSX-002, TSX-007, and TSX-009 formulations were orally administered to female beagle dogs under fasted or fed conditions. Fasted condition means animals were fasted overnight, dosing was performed the subsequent morning, and food was served two hours post-dosing. Animals were allowed access to food for another two hours, and monitored if they ate the food. Fed condition means animals were fasted overnight, dosing was performed the subsequent morning, and food was served 15 min. post-dosing. Animals were allowed access to food for another two hours, and monitored if they ate the food. Blood samples were taken, at 0, 0.5, 1, 2, 3, 4, 6, 8, 12 and 24 hours. The administered dosage of TU was 7.5 mg/kg (TU/body weight). Plasma samples were analyzed on Day 1 and Day 7. Data based on TSX-002, TSX-007, and TSX-009 are reported in FIGS. 2A-2C, respectively. Pharmacokinetic data for treatment days 1 and 7, under fasting and fed conditions are reported for unformulated T in Table 2; unformulated TU in Table 3; TSX-002 (T:Lipid) in Table 4; TSX-007 (native T:Lipid:TPGS (20% w/w of T)) in Table 5; and TSX-009 (TU:Lipid:TPGS (20% w/w of T)) in Table 6.

TABLE 2
pK data for unformulated T.

| | (T) day 1 | (T) day 7 | (T) day 1 | (T) day 7 |
|---|---|---|---|---|
| | fasted | fasted | fed | Fed |
| Parameters | (n = 6) | (n = 6) | (n = 6) | (n = 6) |
| Av. Cmax (ng/mL) | 6.39 ± 2.47 | 2.70 ± 0.78 | 9.85 ± 5.80 | 13.01 ± 13.60 |
| Av. Tmax (h) | 2.17 ± 0.98 | 2.50 ± 1.22 | 3.67 ± 2.58 | 3.5 ± 4.42 |
| Av. AUC (ng · h/mL) | 28.70 ± 9.44 | 11.21 ± 3.28 | 36.84 ± 19.44 | 43 ± 43.91 |

TABLE 3
pK data for unformulated TU.

| | (TU) day 1 | (TU) day 7 | (TU) day 1 | (TU) day 7 |
|---|---|---|---|---|
| | fasted | fasted | fed | Fed |
| Parameters | (n = 6) | (n = 6) | (n = 6) | (n = 6) |
| Av. Cmax (ng/mL) | 2.34 ± 3.94 | 4.38 ± 5.12 | 33.83 ± 5.06 | 5.37 ± 3.78 |
| Av. Tmax (h) | 6.50 ± 4.43 | 8.0 ± 5.66 | 4.0 ± 1.55 | 4.17 ± 3.25 |
| Av. AUC (ng · h/mL) | 16.63 ± 27.15 | 30.88 ± 35.98 | 108.41 ± 42.73 | 20.50 ± 16.59 |

TABLE 4
TSX-002 pK data.

| | (TSX-002) day 1 | (TSX-002) day 7 | (TSX-002) day 1 |
|---|---|---|---|
| | fasted | fasted | fed |
| Parameters | (n = 12) | (n = 12) | (n = 4) |
| Av. Cmax | 5.92 ± 2.18 | 5.13 ± 2.35 | 10.63 ± 3.68 |
| (ng/mL) | | | |
| Av. Tmax | 2.92 ± 1.37 | 2.33 ± 0.65 | 4.25 ± 1.26 |
| (h) | | | |
| Av. AUC | 22.63 ± 7.96 | 16.62 ± 5.63 | 30.0 ± 3.76 |
| (ng · h/mL) | | | |

TABLE 5
TSX-007 pK data.

| | (TSX-007) day 1 | (TSX-007) day 7 | (TSX-007) day 1 |
|---|---|---|---|
| | fasted | fasted | fed |
| Parameters | (n = 12) | (n = 12) | (n = 4) |
| Av. Cmax | 4.06 ± 1.87 | 4.16 ± 1.55 | 11.81 ± 6.16 |
| (ng/mL) | | | |
| Av. Tmax | 2.54 ± 3.06 | 3.75 ± 3.31 | 3.25 ± 0.96 |
| (h) | | | |
| Av. AUC | 20.77 ± 1.82 | 17.30 ± 14.48 | 38.22 ± 15.67 |
| (ng · h/mL) | | | |

TABLE 6
TSX-009 pK data.

| | (TSX-009) day 1 | (TSX-009) day 7 | (TSX-009) day 1 |
|---|---|---|---|
| | fasted | fasted | fed |
| Parameters | (n = 12) | (n = 12) | (n = 4) |
| Av. Cmax | 1.71 ± 2.74 | 5.30 ± 6.00 | 8.39 ± 7.44 |
| (ng/mL) | | | |
| Av. Tmax | 4.67 ± 5.68 | 5.92 ± 5.79 | 4.25 ± 3.50 |
| (h) | | | |
| Av. AUC | 12.39 ± 22.34 | 37.84 ± 46.50 | 37.86 ± 30.46 |
| (ng · h/mL) | | | |

### Example 12

Testosterone Undecanoate+DSPC+Chol.+TPGS+Microcrystalline Cellulose (1.0:0.9:0.1:0.2:0.6) coated dosage form TSX-009. To prepare TSX-009, TU (23.75 g) was dissolved in 100 mL EtOH, at 45-55° C., and mixed until a clear solution formed. DSPC (21.3375 g) and cholesterol (2.375) were added to the drug solution, and the mixture continued to mix at 45-55° C., until a clear solution formed. TPGS (4.75) g was dissolved in 60 ml EtOH separately. Avicel® pH 102 (14.25 g) was added to TPGS solution and dispersed in additional 90 mL EtOH to form a slurry. This slurry was transferred to round bottom flask containing TU, DSPC and cholesterol solution Mixing and heating continued under a vacuum until a dry mass formed which was then milled and screened through a sieve No 60 to obtained dry powder mass. Additional microcrystalline cellulose (113.87 g) (Avicel® PH 102) and sodium starch glycolate (Explotab®) were added to the dry powder, and the combined mixture blended for 20 min using a V blender. The blended mixture was filled into Size “0” Vcaps® Plus capsules to a capsule fill weight of 303.75 mg/capsule, and the capsules were coated with Eudragit® L 30D-55. Table 7 contains the amounts of ingredients per capsule for a TSX-009 formulation containing a 95 mg dose of TU (dose equivalent to 60 mg of T).

TABLE 7
TSX-009 composition.

| | Composition | | |
|---|---|---|---|
| | mg/capsule | wt % | Function |
| Encapsulated Formulation Components | | | |
| Testosterone Undecanoate | 95.0 | 31.27 | Active ingredient |
| DSPC | 85.5 | 28.14 | Phospholipid |
| Cholesterol | 9.5 | 3.12 | Stabilizing agent |
| Vitamin E TPGS | 19 | 6.25 | Antioxidant/Absorption |
| | | | Enhancer |
| Microcrystalline Cellulose NF (Added | 57 | 18.75 | Filler |
| during Matrix preparation stage) | | | |
| Microcrystalline Cellulose NF (Added | 28.75 | 9.46 | Disintegrant |
| during blending stage before | | | |
| encapsulation ) | | | |
| Sodium Starch Glycolate NF | 9.0 | 3.0 | Disintegrant |
| Total of encapsulated components | 303.75 | 100 | |
| Capsule Shell | | | |
| Size 0 Vcaps ® Plus capsule | 1 capsule | — | Encapsulation |
| Enteric Coating composition | | | |
| (Amounts may be approximate.) | | | |
| Methacrylic Acid Copolymer NF, Type C | 34.4 | 62.5 | Coating agent |
| Triethyl Citrate NF | 3.5 | 6.3 | Plasticizer |
| Talc, USP | 17.2 | 31.3 | Glidant |
| Purified Water, USP (Evaporates) | | — | Solvent |
| Total of coating components | 55.1 | 100 | |

### Example 13

Testosterone Undecanoate+DSPC (1.0:1.0), admixed with microcrystalline cellulose at ratio of (Dispersion:Microcrystalline Cellulose) of ratio of 1:3.12, coated dosage form TSX-010 for female dog studies. To prepare TSX-010, TU (11.875 g) was dissolved in 40 mL EtOH, at 45° C.-55, and mixed until a clear solution formed. DSPC (11.875 g) was added to the drug solution, and the mixture continued to mix at 45-55° C., until a clear solution formed. Mixing and heating continued under a vacuum until a dry mass formed which was then milled and screened through a sieve No 60 to obtained dry powder mass. Microcrystalline cellulose (37.80 g) (Avicel® PH 102) and 3.01 g sodium starch glycolate (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55. Table 8 contains the amounts of ingredients per capsule for a TSX-010 formulation containing a 23.8 mg dose of TU (dose equivalent to 15 mg of T).

TABLE 8
TSX-010

| | Composition | | |
|---|---|---|---|
| | mg/capsule | wt % | Function |
| Encapsulated Formulation Components | | | |
| Testosterone Undecanoate | 23.8 | 11.75 | Active ingredient |
| DSPC | 23.8 | 11.75 | Phospholipid |
| Microcrystalline Cellulose NF | 148.9 | 73.51 | Filler |
| Sodium Starch Glycolate NF | 6.0 | 3.0 | Disintegrant |
| Total of encapsulated components | 202.5 | 100 | |
| Capsule Shell | | | |
| Size 0 Vcaps ® Plus capsule | 1 capsule | — | Encapsulation |
| Enteric Coating composition | | | |
| (Amounts may be approximate.) | | | |
| Methacrylic Acid Copolymer NF, Type C | 34.4 | 62.5 | Coating agent |
| Triethyl Citrate NF | 3.5 | 6.3 | Plasticizer |
| Talc, USP | 17.2 | 31.3 | Glidant |
| Purified Water, USP (Evaporates) | | — | Solvent |
| Total of coating components | 55.1 | 100 | |

### Example 14

Testosterone Undecanoate+DSPC (1.0:2.0), admixed with microcrystalline cellulose at ratio of (Dispersion:Microcrystalline Cellulose) of ratio of 1:1.74, coated dosage form TSX-011 for female dog studies. To prepare TSX-011, TU (11.875 g) was dissolved in 60 mL EtOH, at 45-55° C., and mixed until a clear solution formed. DSPC (23.99 g) was added to the drug solution, and the mixture continued to mix at 45-55° C., until a clear solution formed. Mixing and heating continued under a vacuum until a dry mass formed which was then milled and screened through a sieve No 60 to obtained dry powder mass. Microcrystalline cellulose (63.38 g) (Avicel® PH 102) and sodium starch glycolate (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55. Table 9 contains the amounts of ingredients per capsule for a TSX-011 formulation containing a 47.6 mg dose of TU (dose equivalent to 15 mg of T).

TABLE 9
TSX-011 formulation

| | Composition | | |
|---|---|---|---|
| | mg/capsule | wt % | Function |
| Encapsulated Formulation Components | | | |
| Testosterone Undecanoate | 23.8 | 11.7 | Active ingredient |
| DSPC | 47.6 | 23.5 | Phospholipid |
| Microcrystalline Cellulose NF | 124.93 | 61.7 | Filler |
| Sodium Starch Glycolate NF | 6.07 | 3.0 | Disintegrant |
| Total of encapsulated components | 202.5 | 100 | |
| Capsule Shell | | | |
| Size 0 Vcaps ® Plus capsule | 1 capsule | — | Encapsulation |
| Enteric Coating composition | | | |
| (Amounts may be approximate) | | | |
| Methacrylic Acid Copolymer NF, Type C | 34.4 | 62.5 | Coating agent |
| Triethyl Citrate NF | 3.5 | 6.3 | Plasticizer |
| Talc, USP | 17.2 | 31.3 | Glidant |
| Purified Water, USP (Evaporates) | | — | Solvent |
| Total of coating components | 55.1 | 100 | |

### Example 15

Testosterone Undecanoate+DSPC (1.0:4.0), admixed with microcrystalline cellulose at TU:MC ratio of 1:0.65, coated dosage form TSX-012. To prepare TSX-012, TU (11.875 g) was dissolved in 120 mL EtOH, at 45-55° C., and mixed until a clear solution formed. DSPC (147.5 g) was added to the drug solution, and the mixture continued to mix at 45-55° C., until a clear solution formed. Mixing and heating continued under a vacuum until a dry mass formed which was then milled and screened through a sieve No 60 to obtained dry powder mass. Microcrystalline cellulose (33.39 g) (Avicel® PH 102) and 2.96 g sodium starch glycolate (Explotab®) were added to the dry powder, and the combined mixture blended for 20 min using a V blender. The blended mixture was filled into Size “1” Vcaps® Plus capsules to a capsule fill weight of 202.5 mg/capsule, and the capsules were coated with Eudragit® L 30D-55. Table 10 contains the amounts of ingredients per capsule for a TSX-011 formulation containing a 23.8 mg dose of TU (dose equivalent to 15 mg of T).

TABLE 10
TSX-012 composition.

| | Composition | | |
|---|---|---|---|
| | mg/capsule | wt % | Function |
| Encapsulated Formulation Components | | | |
| Testosterone Undecanoate | 23.8 | 11.75 | Active ingredient |
| DSPC | 95.2 | 47.01 | Phospholipid |
| Microcrystalline Cellulose NF | 77.425 | 38.23 | Filler |
| Sodium Starch Glycolate NF | 6.07 | 3.00 | Disintegrant |
| Total of encapsulated components | 202.5 | 100 | |
| Capsule Shell | | | |
| Size 0 Vcaps ® Plus capsule | 1 capsule | — | Encapsulation |
| Enteric Coating composition | | | |
| (Amounts may be approximate) | | | |
| Methacrylic Acid Copolymer NF, Type C | 34.4 | 62.5 | Coating agent |
| Triethyl Citrate NF | 3.5 | 6.3 | Plasticizer |
| Talc, USP | 17.2 | 31.3 | Glidant |
| Purified Water, USP (Evaporates) | | — | Solvent |
| Total of coating components | 55.1 | 100 | |

### Example 16

Plasma testosterone concentrations after administration of TSX-010 (TU:Lipid, 1:1), TSX-011 (TU:Lipid, 1:2), and TSX-012 (TU:Lipid, 1:4). Unformulated TU, TSX-010, TSX-011, and TSX-012 formulations were orally administered under either fasted or fed conditions. The fasted condition means animals were fasted overnight, dosing was performed the subsequent morning, and food was served two hours post-dosing. Animals were allowed access to food for another two hours, and monitored if they ate the food. The fed condition means animals were fasted overnight, dosing was performed the subsequent morning, and food was served 15 min. post-dosing. Animals were allowed access to food for another two hours, and monitored if they ate the food. For unformulated TU, blood samples were taken by venipuncture of the jugular vein at 0, 0.5, 1, 2, 3, 4, 6, 8, 12, and 24 hours in both fed and fasted conditions. For TU formulations like TSX-010, TSX-011, TSX-012, blood samples were taken by venipuncture of the jugular vein at 0, 4, 6, 8, 10, 12, 14, 16, 18 and 24 hours in fasted conditions and at 0, 1, 2, 4, 6, 8, 10, 12, 16 and 24 hours in fed conditions in femalebeagle dogs. The administered dosages of TU were either 1.875, 3.75, or 7.5 mg/kg (TU/body weight). Plasma testosterone concentration data, was obtained at day 1 for formulations TSX-010, TSX-011, and TSX-012, and days 1 and 7, for unformulated TU. An animal was considered a non-responder if its plasma testosterone level did not exceed a 0.5 ng/mL quantification limit. Table 11 shows the fraction of animals that responded to the foregoing TU formulation treatments within a 24 hour time period.

TABLE 11

| | Administered | | Animals with plasma | |
|---|---|---|---|---|
| TU Formulation | TU (mg/kg) | Fasting/Fed | T > 0.5 ng/mL | |
| Unformulated TU | 7.5 | Fasted, d 1 | 2/6 | (33.3%) |
| Unformulated TU | 7.5 | Fed, d 1 | 6/6 | (100%) |
| Unformulated TU | 7.5 | Fasted, d 7 | 3/6 | (50%) |
| Unformulated TU | 7.5 | Fed, d 7 | 5/6 | (100%) |
| TSX-010 TU:DSPC (1.0:1.0) | 1.875 | Fasted | 1/4 | (25%) |
| TSX-010 | 1.875 | Fed | 3/4 | (75%) |
| TSX-010 | 3.75 | Fasted | 1/4 | (25%) |
| TSX-010 | 3.75 | Fed | 3/4 | (75%) |
| TSX-010 | 7.5 | Fasted | 1/4 | (25%) |
| TSX-010 | 7.5 | Fed | 4/4 | (100%) |
| TSX-011 TU:DSPC (1.0:2.0) | 1.875 | Fasted | 2/4 | (50%) |
| TSX-011 | 1.875 | Fed | 2/4 | (50%) |
| TSX-011 | 3.75 | Fasted | 2/4 | (50%) |
| TSX-011 | 3.75 | Fed | 3/4 | (75%) |
| TSX-011 | 7.5 | Fasted | 3/4 | (75%) |
| TSX-011 | 7.5 | Fed | 4/4 | (100%) |
| TSX-012 TU:DSPC (1.0:4.0) | 1.875 | Fasted | 2/4 | (50%) |
| TSX-012 | 1.875 | Fed | 3/4 | (75%) |
| TSX-012 | 3.75 | Fasted | 4/4 | (100%) |
| TSX-012 | 3.75 | Fed | 4/4 | (100%) |
| TSX-012 | 7.5 | Fasted | 2/4 | (50%) |
| TSX-012 | 7.5 | Fed | 4/4 | (100%) |

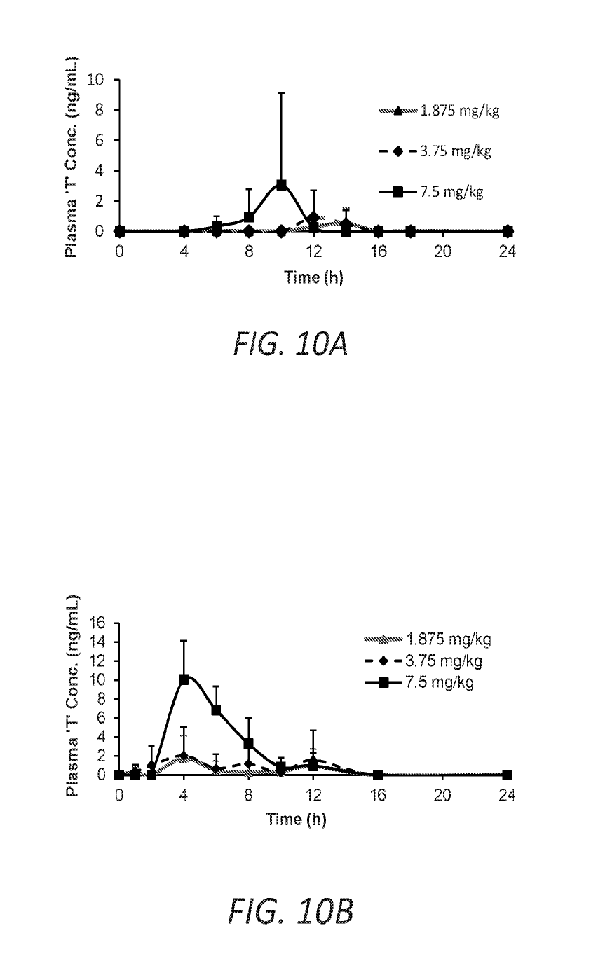

Tables 12 and 13 report plasma T concentrations at each time point for formulation TSX-010, and these data are represented graphically in FIGS. 10A and 10B.

TABLE 12

| | Plasma T (ng/mL) | | | | | |
|---|---|---|---|---|---|---|
| | Mean levels indicative of response are in bold | | | | | |
| Hours post-TSX-010 | 1.875 mg/kg | | 3.75 mg/kg | | 7.5 mg/kg | |
| administration under | Mean | | Mean | | Mean | |
| fasted conditions | (n = 4) | SD | (n = 4) | SD | (n = 4) | SD |
| 0 | 0.00 | 0.00 | 0.00 | 0.00 | 0.00 | 0.00 |
| 4 | 0.00 | 0.00 | 0.00 | 0.00 | 0.00 | 0.00 |
| 6 | 0.00 | 0.00 | 0.00 | 0.00 | 0.33 | 0.67 |
| 8 | 0.00 | 0.00 | 0.00 | 0.00 | 0.93 | 1.86 |
| 10 | 0.00 | 0.00 | 0.00 | 0.00 | 3.05 | 6.10 |
| 12 | 0.31 | 0.61 | 0.91 | 1.81 | 0.26 | 0.52 |
| 14 | 0.51 | 1.02 | 0.46 | 0.92 | 0.00 | 0.00 |
| 16 | 0.00 | 0.00 | 0.00 | 0.00 | 0.00 | 0.00 |
| 18 | 0.00 | 0.00 | 0.00 | 0.00 | 0.00 | 0.00 |
| 24 | 0.00 | 0.00 | 0.00 | 0.00 | 0.00 | 0.00 |

TABLE 13

| | Plasma T (ng/mL) | | | | | |
|---|---|---|---|---|---|---|
| | Mean levels indicative of response are in bold | | | | | |
| Hours post-TSX-010 | 1.875 mg/kg | | 3.75 mg/kg | | 7.5 mg/kg | |
| administration under | Mean | | Mean | | Mean | |
| fed conditions | (n = 4) | SD | (n = 4) | SD | (n = 4) | SD |
| 0 | 0.00 | 0.00 | 0.00 | 0.00 | 0.00 | 0.00 |
| 1 | 0.29 | 0.57 | 0.37 | 0.75 | 0.00 | 0.00 |
| 2 | 0.00 | 0.00 | 1.02 | 2.04 | 0.00 | 0.00 |
| 4 | 1.80 | 2.37 | 2.06 | 3.01 | 10.06 | 4.11 |
| 6 | 0.50 | 1.01 | 0.73 | 1.46 | 6.86 | 2.51 |
| 8 | 0.35 | 0.69 | 1.19 | 2.38 | 3.30 | 2.75 |
| 10 | 0.37 | 0.75 | 0.26 | 0.52 | 0.84 | 0.98 |
| 12 | 0.92 | 1.84 | 1.58 | 3.16 | 1.01 | 1.34 |
| 16 | 0.00 | 0.00 | 0.00 | 0.00 | 0.00 | 0.00 |
| 24 | 0.00 | 0.00 | 0.00 | 0.00 | 0.00 | 0.00 |

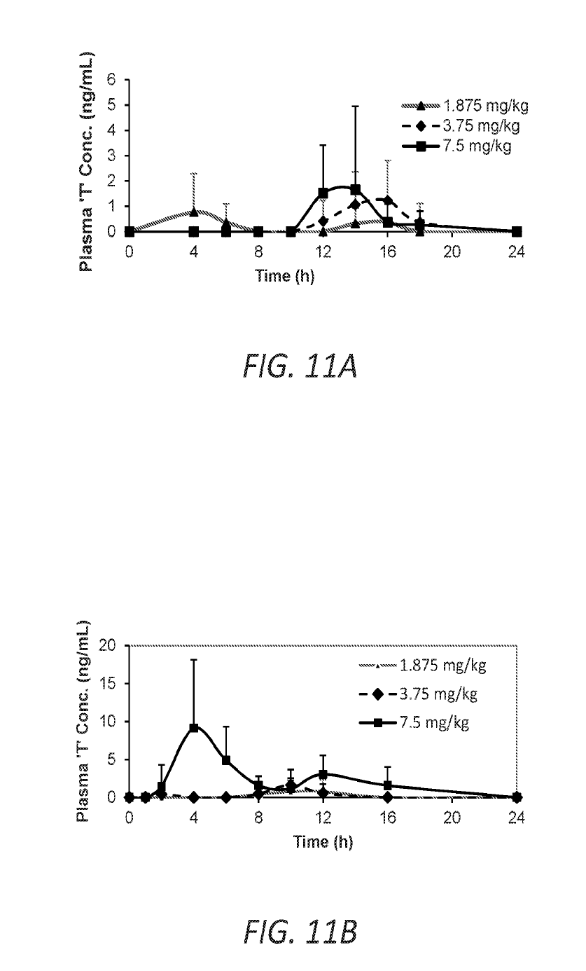

Tables 14 and 15 report plasma T concentrations at each time point for formulation TSX-011, and these data are represented graphically in FIGS. 11A and 11B.

TABLE 14

| | Plasma T (ng/mL) | | | | | |
|---|---|---|---|---|---|---|
| | Mean levels indicative of response are in bold | | | | | |
| Hours post-TSX-011 | 1.875 mg/kg | | 3.75 mg/kg | | 7.5 mg/kg | |
| administration under | Mean | | Mean | | Mean | |
| fasted conditions | (n = 4) | SD | (n = 4) | SD | (n = 4) | SD |
| 0 | 0.00 | 0.00 | 0.00 | 0.00 | 0.00 | 0.00 |
| 4 | 0.76 | 1.53 | 0.00 | 0.00 | 0.00 | 0.00 |
| 6 | 0.37 | 0.73 | 0.00 | 0.00 | 0.00 | 0.00 |
| 8 | 0.00 | 0.00 | 0.00 | 0.00 | 0.00 | 0.00 |
| 10 | 0.00 | 0.00 | 0.00 | 0.00 | 0.00 | 0.00 |
| 12 | 0.00 | 0.00 | 0.41 | 0.82 | 1.51 | 1.90 |
| 14 | 0.32 | 0.63 | 1.07 | 1.30 | 1.65 | 3.31 |
| 16 | 0.39 | 0.77 | 1.23 | 1.58 | 0.37 | 0.73 |
| 18 | 0.00 | 0.00 | 0.37 | 0.74 | 0.27 | 0.54 |
| 24 | 0.00 | 0.00 | 0.00 | 0.00 | 0.00 | 0.00 |

TABLE 15

| Hours | Plasma T (ng/mL) | | | | | |
|---|---|---|---|---|---|---|
| post-TSX-011 | Mean levels indicative of response are in bold | | | | | |
| administration | 1.875 mg/kg | | 1.875 mg/kg | | 1.875 mg/kg | |
| under fed | Mean | Mean | Mean | Mean | Mean | Mean |
| conditions | (n = 4) | (n = 4) | (n = 4) | (n = 4) | (n = 4) | (n = 4) |
| 0 | 0.00 | 0.00 | 0.00 | 0.00 | 0.00 | 0.00 |
| 1 | 0.00 | 0.00 | 0.00 | 0.00 | 0.00 | 0.00 |
| 2 | 0.00 | 0.00 | 0.43 | 0.85 | 1.44 | 2.89 |
| 4 | 0.00 | 0.00 | 0.00 | 0.00 | 9.13 | 9.00 |
| 6 | 0.00 | 0.00 | 0.00 | 0.00 | 4.87 | 4.42 |
| 8 | 0.43 | 0.86 | 0.46 | 0.92 | 1.58 | 1.20 |
| 10 | 0.76 | 1.52 | 1.64 | 2.04 | 1.16 | 1.38 |
| 12 | 0.77 | 1.54 | 0.59 | 1.17 | 3.04 | 2.48 |
| 16 | 0.00 | 0.00 | 0.00 | 0.00 | 1.57 | 2.45 |
| 24 | 0.00 | 0.00 | 0.00 | 0.00 | 0.00 | 0.00 |

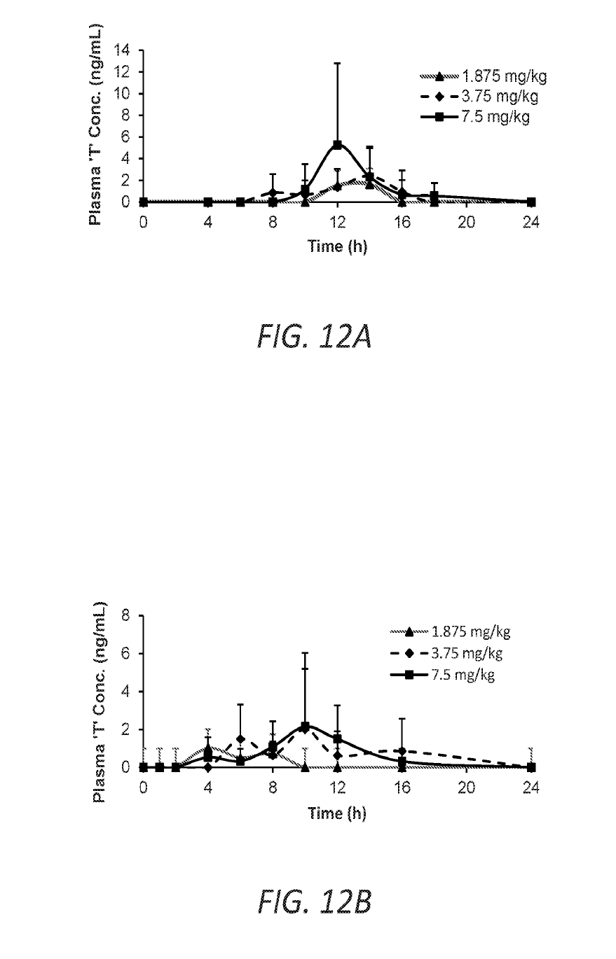

Tables 16 and 17 report plasma T concentrations at each time point for formulation TSX-012, and these data are represented graphically in FIGS. 12A and 12B.

TABLE 16

| | Plasma T (ng/mL) | | | | | |
|---|---|---|---|---|---|---|
| | Mean levels indicative of response are in bold | | | | | |
| Hours post-TSX-012 | 1.875 mg/kg | | 3.75 mg/kg | | 7.5 mg/kg | |
| administration under | Mean | | Mean | | Mean | |
| fasted conditions | (n = 4) | SD | (n = 4) | SD | (n = 4) | SD |
| 0 | 0.00 | 0.00 | 0.00 | 0.00 | 0.00 | 0.00 |
| 4 | 0.00 | 0.00 | 0.00 | 0.00 | 0.00 | 0.00 |
| 6 | 0.00 | 0.00 | 0.00 | 0.00 | 0.00 | 0.00 |
| 8 | 0.00 | 0.00 | 0.86 | 1.71 | 0.00 | 0.00 |
| 10 | 0.00 | 0.00 | 0.66 | 1.32 | 1.17 | 2.34 |
| 12 | 1.52 | 1.37 | 1.37 | 1.67 | 5.25 | 7.54 |
| 14 | 1.64 | 1.47 | 2.42 | 2.55 | 2.31 | 2.80 |
| 16 | 0.00 | 0.00 | 0.97 | 1.94 | 0.67 | 1.34 |
| 18 | 0.00 | 0.00 | 0.00 | 0.00 | 0.58 | 1.16 |
| 24 | 0.00 | 0.00 | 0.00 | 0.00 | 0.00 | 0.00 |

TABLE 17

| | Plasma T (ng/mL) | | | | | |
|---|---|---|---|---|---|---|
| Hours post-TSX-012 | Mean levels indicative of response are in bold | | | | | |
| administration | 1.875 mg/kg | | 3.75 mg/kg | | 7.5 mg/kg | |
| under fed | Mean | | Mean | | Mean | |
| conditions | (n = 4) | SD | (n = 4) | SD | (n = 4) | SD |
| 0 | 0.00 | 0.00 | 0.00 | 0.00 | 0.00 | 0.00 |
| 1 | 0.00 | 0.00 | 0.00 | 0.00 | 0.00 | 0.00 |
| 2 | 0.00 | 0.00 | 0.00 | 0.00 | 0.00 | 0.00 |
| 4 | 1.02 | 2.04 | 0.00 | 0.00 | 0.53 | 1.06 |
| 6 | 0.50 | 1.01 | 1.50 | 1.81 | 0.33 | 0.66 |
| 8 | 0.74 | 1.47 | 0.64 | 0.74 | 1.08 | 1.36 |
| 10 | 0.00 | 0.00 | 2.01 | 4.02 | 2.15 | 3.04 |
| 12 | 0.00 | 0.00 | 0.63 | 1.27 | 1.50 | 1.77 |
| 16 | 0.00 | 0.00 | 0.86 | 1.72 | 0.31 | 0.63 |
| 24 | 0.00 | 0.00 | 0.00 | 0.00 | 0.00 | 0.00 |

### Example 17

In vivo pK data for TSX-010 (TU:Lipid, 1:1), TSX-011 (TU:Lipid, 1:2), and TSX-012 (TU:Lipid, 1:4) in female Beagle dogs. Unformulated TU, TSX-010, TSX-011, and TSX-012 formulations. In addition to tracking plasma T levels, blood samples taken by jugular vein puncture at 0, 0.5, 1, 2, 3, 4, 6, 8, 12, 24 hours, and the following pharmacokinetic (PK) parameters were analyzed for each formulation and unformulated testosterone and testosterone undecanoate. Table 18 contains PK data for unformulated testosterone and testosterone undecanoate at days 1 and 7, under fasted and fed conditions. Tables 19 and 20 contain PK data for the TSX-010, TSX-011, and TSX-012 formulations under fasted (Table 19) and fed (Table 20) conditions.

Unformulated TU showed better absorption under fed conditions as compared to fasted state, as evidenced by a two-fold increase in AUC and Cmax in the presence of food. However, on Day 7 after administration of unformulated TU, plasma profiles dropped significantly in comparison to animals which responded to the treatment with proliposomal formulations of TU. TU formulations, TSX-010, -011, and -012 were tested in female beagle dogs to identify optimal TU to DSPC ratios and TU dosages, for further studies to performed using male dogs. Three dosage amounts were tested (1.87, 3.75, and 7.5 mg/Kg). Plasma levels were relatively high following administration of the 7.5 mg/Kg dose under fed and fasted conditions for all TSX-010, TSX-011, TSX-012.

TSX-011, which has a TU to DSPC ratio (w/w) of 1.0:2.0, was associated with higher TU absorption, followed by that of TSX-010 under fed conditions. There were no supra physiological levels of “T” in the presence of food for any of the TU formulations. However, TU formulated as TSX-012, did not absorb well under fed conditions, despite its higher TU to DSPC ratio of 1.0:4.0, but it was associated with relatively high AUC values under fasted conditions. TSX-011 with drug to lipid ratio of 1.0:2.0 had high responders with minimum variability and hence was selected to for further evaluation in male beagle dogs.

TABLE 18
PK data for unformulated T and TU.

| | Unformulated T | | Unformulated TU | | Unformulated T | | Unformulated TU | |
|---|---|---|---|---|---|---|---|---|
| | Fasted | | fasted | | fed | | fed | |
| | Administration Day | | | | | | | |
| | Day 1 | Day 7 | Day 1 | Day 7 | Day 1 | Day 7 | Day 1 | Day 7 |
| Parameters | (n = 6) | (n = 6) | (n = 6) | (n = 6) | (n = 6) | (n = 6) | (n = 6) | (n = 6) |
| Av. Cmax | 6.39 ± 2.47 | 2.70 ± 0.78 | 2.34 ± 3.94 | 4.38 ± 5.12 | 9.85 ± 5.80 | 13.01 ± 13.60 | 33.83 ± 5.06 | 5.37 ± 3.78 |
| (ng/mL) | | | | | | | | |
| Av. Tmax (h) | 2.17 ± 0.98 | 2.50 ± 1.22 | 6.50 ± 4.43 | 8.0 ± 5.66 | 3.67 ± 2.58 | 3.5 ± 4.42 | 4.0 ± 1.55 | 4.17 ± 3.25 |
| Av. AUC | 28.70 ± 9.44 | 11.21 ± 3.28 | 16.63 ± 27.15 | 30.88 ± 35.98 | 36.84 ± 19.44 | 43 ± 43.91 | 108.41 ± 42.73 | 20.50 ± 16.59 |
| (ng · h/mL) | | | | | | | | |

TABLE 19
Comparative PK data for TSX-010, TSX-011, and TSX-012 under fasted conditions.

| | 1.875 mg/kg | | | 3.75 mg/kg | | | 7.5 mg/kg | | |
|---|---|---|---|---|---|---|---|---|---|
| | Day 1, Fasted | | | Day 1, Fasted | | | Day 1, Fasted | | |
| | (n = 4) | | | (n = 4) | | | (n = 4) | | |
| Para- | Formulation | | | | | | | | |
| meters | TSX-010 | TSX-011 | TSX-012 | TSX-010 | TSX-011 | TSX-012 | TSX-010 | TSX-011 | TSX-012 |
| Av. Cmax | 0.51 ± 1.02 | 1.15 ± 1.46 | 2.18 ± 1.20 | 0.91 ± 1.81 | 1.23 ± 1.58 | 3.85 ± 1.39 | 3.05 ± 6.10 | 2.45 ± 2.90 | 5.41 ± 7.54 |
| (ng/mL) | | | | | | | | | |
| Av. Tmax | 14 | 10.0 ± 8.49 | 13.33 ± 1.15 | 12 | 16 ± 0 | 12.0 ± 2.83 | 10 | 14.67 ± 3.06 | 13.0 ± 1.41 |
| (h) | (n = 1) | | | (n = 1) | | | (n = 1) | | |
| Av. AUC | 1.63 ± 3.26 | 4.42 ± 5.74 | 6.30 ± 5.67 | 2.73 ± 5.46 | 6.9 ± 9.97 | 12.53 ± 4.83 | 9.14 ± 18.27 | 8.14 ± 10.76 | 21.11 ± 24.91 |
| (ng · | | | | | | | | | |
| h/mL) | | | | | | | | | |

TABLE 20
Comparative PK data for TSX-010, TSX-011, and TSX-012 under fed conditions.

| | 1.875 mg/kg | | | 3.75 mg/kg | | | 7.5 mg/kg | | |
|---|---|---|---|---|---|---|---|---|---|
| | Day 1, Fed | | | Day 1, Fed | | | Day 1, Fed | | |
| | (n = 4) | | | (n = 4) | | | (n = 4) | | |
| Para- | Formulation | | | | | | | | |
| meters | TSX-010 | TSX-011 | TSX-012 | TSX-010 | TSX-011 | TSX-012 | TSX-010 | TSX-011 | TSX-012 |
| Av. Cmax | 2.72 ± 2.15 | 1.53 ± 1.77 | 1.75 ± 2.08 | 4.36 ± 3.0 | 2.07 ± 1.74 | 4.07 ± 2.87 | 10.49 ± 3.71 | 10.74 ± 7.21 | 3.15 ± 2.24 |
| (ng/mL) | | | | | | | | | |
| Av. Tmax | 6.67 ± 4.62 | 11.0 ± 1.41 | 6.0 ± 2.83 | 8.0 ± 4.0 | 7.33 ± 4.62 | 10.0 ± 4.32 | 4.5 ± 1.0 | 6.5 ± 3.79 | 8.5 ± 3.42 |
| (h) | | | | | | | | | |
| Av. AUC | 9.08 ± 7.02 | 4.69 ± 5.41 | 4.51 ± 5.80 | 15.09 ± 10.10 | 6.59 ± 8.56 | 15.34 ± 12.33 | 45.15 ± 12.37 | 54.12 ± 20.9 | 14.53 ± 10.75 |
| (ng · | | | | | | | | | |
| h/mL) | | | | | | | | | |

### Example 18

Testosterone Undecanoate+DSPC (1.0:2.0), admixed with microcrystalline cellulose at ratio of (Dispersion: Microcrystalline Cellulose) of ratio of 1:1.06, coated dosage form TSX-011 in male beaglgs. TSX-011 was prepared as capsule containing TU Eq to 30 mg of T per capsules for studies in male beagle dogs. To prepare TSX-011, TU (35.70 g) was dissolved in 171 mL EtOH, at 45-55° C., and mixed until a clear solution formed. DSPC (71.40 g) was added to the drug solution, and the mixture continued to mix at 45-55° C., until a clear solution formed. Mixing and heating continued under a vacuum until a dry mass formed which was then milled and screened through a sieve No 60 to obtained dry powder mass. Microcrystalline cellulose (113.87 g) (Avicel® PH 102) and sodium starch glycolate (Explotab®) were added to the dry powder, and the combined mixture blended for 20 min using a V blender. The blended mixture was filled into Size “0” Vcaps® Plus capsules to a capsule fill weight of 303.75 mg/capsule, and the capsules were coated with Eudragit® L 30D-55. Table 21 contains the amounts of ingredients per capsule for a TSX-011 formulation containing a 47.6 mg dose of TU (dose equivalent to 30 mg of T).

TABLE 21
TSX-011 formulation

| | Composition | | |
|---|---|---|---|
| | mg/capsule | wt % | Function |
| Encapsulated Formulation Components | | | |
| Testosterone Undecanoate | 47.60 | 15.67 | Active ingredient |
| DSPC | 95.20 | 31.35 | Phospholipid |
| Microcrystalline Cellulose NF | 151.80 | 49.98 | Filler |
| Sodium Starch Glycolate NF | 9.10 | 3.00 | Disintegrant |
| Total of encapsulated components | 303.7 | 100 | |
| Capsule Shell | | | |
| Size 0 Vcaps ® Plus capsule | 1 capsule | — | Encapsulation |
| Enteric Coating composition | | | |
| (Amounts may be approximate) | | | |
| Methacrylic Acid Copolymer NF, Type C | 34.4 | 62.5 | Coating agent |
| Triethyl Citrate NF | 3.5 | 6.3 | Plasticizer |
| Talc, USP | 17.2 | 31.3 | Glidant |
| Purified Water, USP (Evaporates) | | — | Solvent |
| Total of coating components | 55.1 | 100 | |

### Example 19

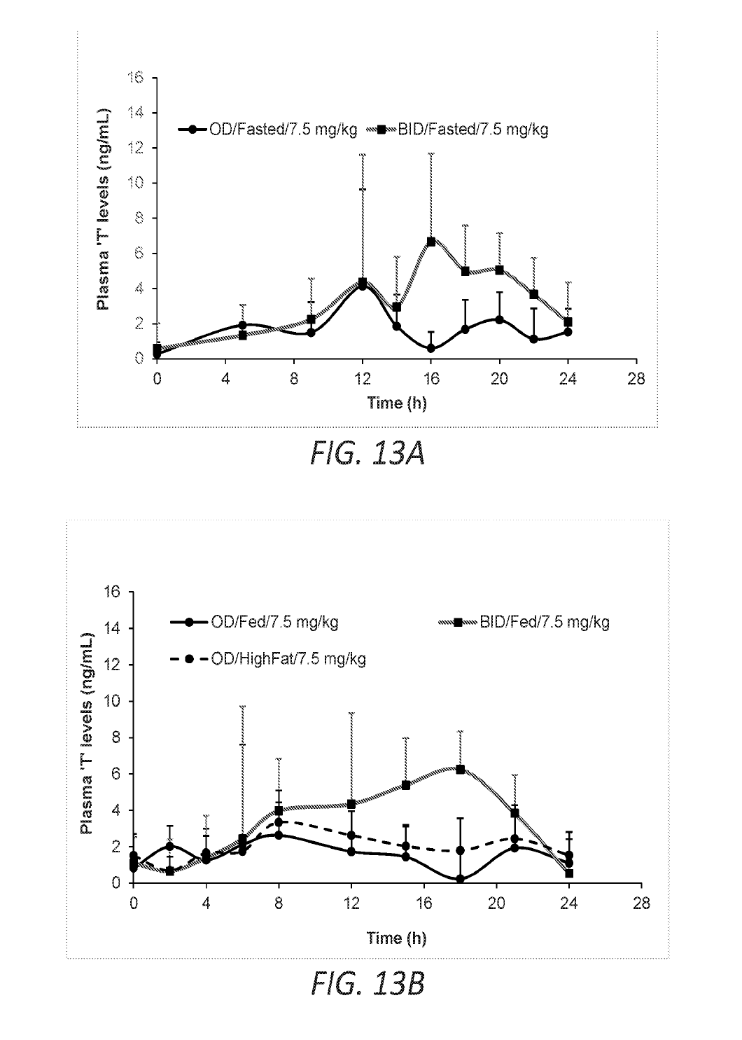

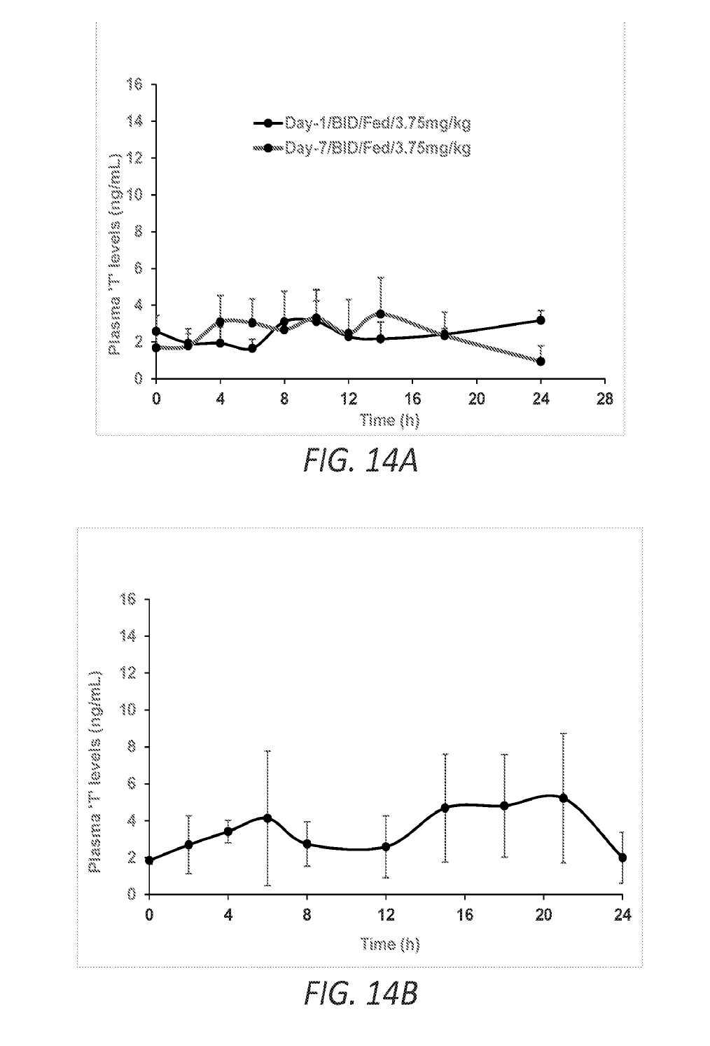

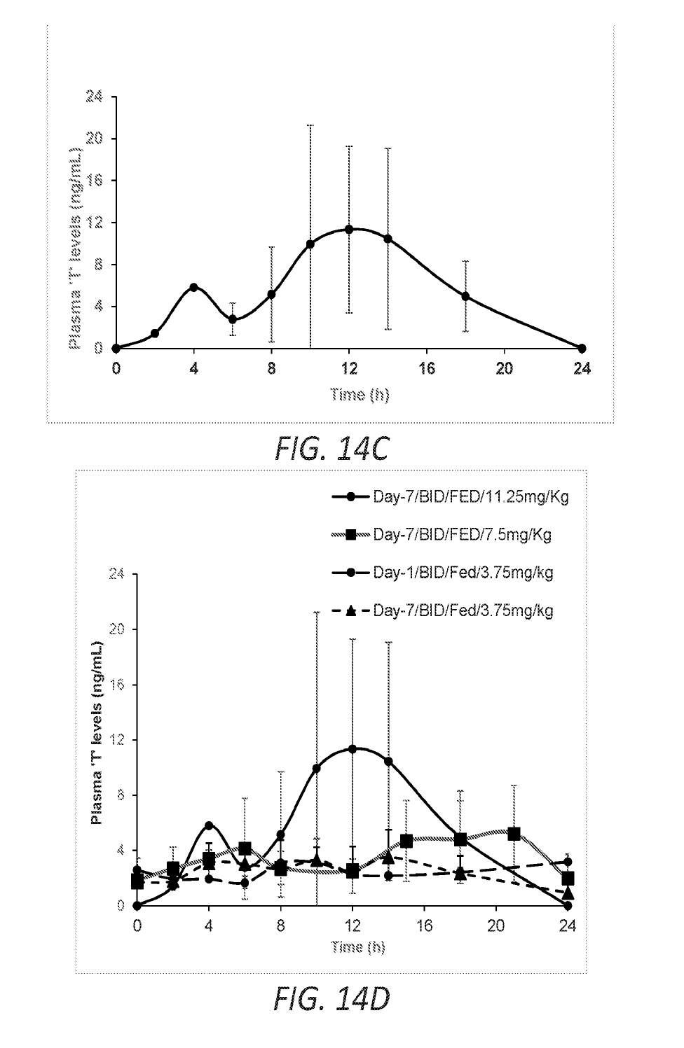

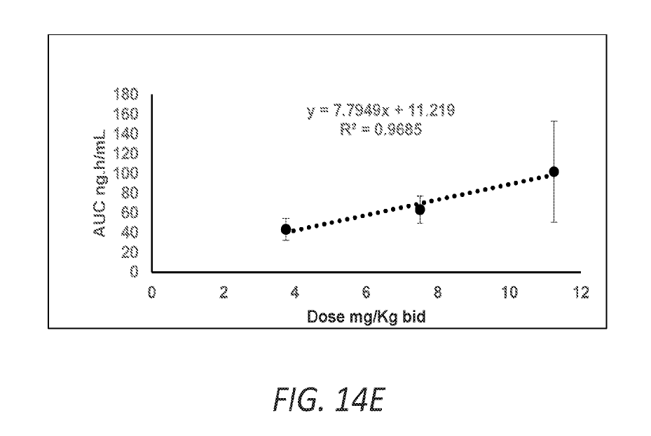

Plasma testosterone concentrations after administration of TSX-011 (TU:Lipid, 1:2) to male Beagle dogs. TSX-011 was orally administered under either fasted or fed conditions in male beagle dogs. The fasted condition means animals were fasted overnight, dosing was performed the subsequent morning, and food was served two hours post-dosing. Animals were allowed access to food for another two hours, and monitored if they ate the food. The fed condition means animals were fasted overnight, dosing was performed the subsequent morning, and food was served 15 min. post-dosing. A study was also performed with high fat meals containing 21% fat based on dry basis and 41% fat based on calories. Animals were fasted overnight & the food was offered approximately 15 to 30 minutes after dosing. The feed was made available for a period of approximately 4 hours and then removed. A 300 gram of food was provided once daily/split in two equal portions of 150 grams For once a day dosing, blood samples were taken by venipuncture of the jugular vein at 0, 5, 9, 12, 14, 16, 18, 20, and 24 hours under fasting conditions and under fed conditions blood samples were collected at 0, 2, 4, 6, 8, 12, 14, 18, 22 and 24 hours including the study with high fat meal. The administered dosages of TU Eq to T from TSX-011 were either 7.5 mg/kg (TU Eq to T/body weight) once a day or twice a day. Plasma testosterone concentration data, for TSX-011 under fasted/fed conditions are compiled in Table 22, 23 and the same data is graphically represented in FIGS. 13A, 13B. Increasing the dose from QD to BID enhanced the plasma profile of TSX-011 both in fed and fasted conditions. However, presence of high fat did not show any supra physiological levels of T with TSX-011. The food effect was minimum as the difference in the AUC of QD/Fed and QD/Fed/High fat was not significant.

TABLE 22
Comparative PK data for TSX-011 under fasted
conditions with two different dosings.

| Parameters | QD/Fasted | BID/Fasted |
|---|---|---|
| Av. Cmax (ng/mL) | 5.43 ± 4.73 | 10.82 ± 5.26 |
| Av. Tmax (h) | 13.83 ± 5.81 | 15.66 ± 3.67 |
| Av. AUC (ng · h/mL) | 41.35 ± 25.68 | 74.98 ± 21.93 |

TABLE 23
Comparative PK data of TSX-011 under fed
conditions with two different dosings.

| Parameters | QD/Fed | QD/Fed/High Fat | BID/Fed |
|---|---|---|---|
| Av. Cmax | 5.85 ± 2.79 | 4.62 ± 1.32 | 10.38 ± 4.21 |
| (ng/mL) | | | |
| Av. Tmax | 9.5 ± 4.54 | 14.17 ± 6.77 | 11.67 ± 3.88 |
| (h) | | | |
| Av. AUC | 37.99 ± 13.64 | 50.01 ± 12.68 | 76.98 ± 29.36 |
| (ng · h/mL) | | | |

### REFERENCES

Yin, A et al. “Dietary Fat Modulates the Testosterone Pharmacokinetics of a New Self-Emulsifying Formulation of Oral Testosterone Undecanoate in Hypogonadal Men.” J. of Androl. 33:1282-1290. (2012).

1. A proliposomal powder dispersion comprising
(a) testosterone undecanoate (TU) and
(b) distearoyl phosphatidylcholine (DSPC),
wherein the (TU) and (DSPC) are present in the dispersion in a weight/weight (w/w) ratio of (a):(b), that ranges from (1.0:1.0) to (1.0:4.0).

2. proliposomal powder dispersion according to claim 1, wherein the w/w ratio (a):(b) is (1.0:2.0).

3. oral dosage form comprising a proliposomal powder dispersion according to claim 1.

4. oral dosage form according to claim 3, additionally comprising at least one pharmaceutically acceptable excipient.

5. oral dosage form according to claim 4, wherein the at least one pharmaceutically acceptable excipient is microcrystalline cellulose or sodium starch glycolate or both.

6. oral dosage form according to claim 5, comprising microcrystalline cellulose, wherein the proliposomal powder dispersion and microcrystalline cellulose are present in a w/w ratio that ranges from (1.0:1.00) to (1.0:1.40), or any ratio therein.

7. oral dosage form according to claim 5, comprising sodium starch glycolate, wherein the proliposomal powder dispersion and sodium starch glycolate are present in a w/w ratio that ranges from (1.0:0.050) to (1.0:0.80),or any ratio therein.

8. oral dosage form according to claim 5, comprising microcrystalline cellulose and sodium starch glycolate, wherein the proliposomal powder dispersion and microcrystalline cellulose are present in a w/w ratio of (1.0:1.06), and wherein the proliposomal powder dispersion and sodium starch glycolate are present in a w/w ratio of (1.0:0.064) to (1.0:1.10).

9. oral dosage form according to claim 5, wherein the dosage form is a capsule.

10. oral dosage form according to claim 9, wherein the capsule is coated with an enteric coating composition.

11. oral dosage form according to claim 10, wherein the coating composition comprises a methacrylic acid copolymer.

12. A method of testosterone replacement therapy (TRT) for an individual in need thereof, comprising administering an oral dosage form according to claim 5.

13. The method of TRT according to claim 12, wherein the individual is in need of TRT to treat low testosterone levels resulting from at least one of the following conditions: a consequence of injury; infection; loss of the testicles; chemotherapy; radiation treatment; genetic abnormalities; hemochromatosis; dysfunction of the pituitary gland; inflammatory disease; medication side effect; chronic kidney failure; liver cirrhosis, stress; alcoholism; obesity; Kallman's syndrome; male hypogonadism; and testosterone deficiency syndrome (TDS).

14. The method of TRT according to claim 12, wherein the pre-therapy serum testosterone concentration of the individual in need thereof is less than 300 ng/dL.

15. The method of TRT according to claim 12, wherein the clinical effectiveness of the dosage form is independent of food effects.
